(19) United States
(12) Patent Application Publication
(10) Pub. No.: US 2023/0302235 A1
(43) Pub. Date: Sep. 28, 2023

(54) INHALER SYSTEM

(71) Applicant: Norton (Waterford) Limited, Waterford (IE)

(72) Inventors: Mark Milton-Edwards, Chesire (GB); Guilherme Safioti, Stigtomta (CH)

(73) Assignee: Norton (Waterford) Limited, Waterford (IE)

(21) Appl. No.: 18/019,408

(22) PCT Filed: Aug. 6, 2021
(86) PCT No.: PCT/EP2021/072040

§ 371 Date: Feb. 2, 2023

(30) Foreign Application Priority Data
Aug. 6, 2020 (GB) 2012249.5
Aug. 6, 2020 (GB) 2012251.1

(51) Int. Cl. A61M 15/00; G16H 20/13; G16H 50/30; G16H 40/67

(52) U.S. Cl. CPC A61M 15/0001; G16H 20/13; G16H 50/30; G16H 40/67; A61M 2205/502; A61B 2010/0087

(57) ABSTRACT

Provided is a system comprising an inhaler. The inhaler comprises a use determination system. The use determination system is configured to determine at least one first value of a usage parameter relating to use of the inhaler. A processing module is configured to receive the at least one first value. The processing module is also configured to receive a second value indicative of whether the subject is also suffering from a viral respiratory infection. The processing module then determines the level of acute risk based on the at least one first value and the second value. Also provided is a system for determining a probability that a subject having a chronic respiratory disease also has a severe acute respiratory syndrome. The system comprises a first inhaler for delivering a rescue medicament prescribed for the chronic respiratory disease. The first inhaler has a use determination system configured to determine a rescue inhalation performed by the subject using the first inhaler. The system optionally includes a second inhaler for delivering a maintenance medicament to the subject during a routine inhalation A sensor system is configured to measure a parameter relating to airflow during the rescue inhalation and/or during the routine inhalation, when the second inhaler is included in the system. The system further comprises a processor configured to determine a number of the rescue inhalations during a first time period, and receive the parameter measured for at least some of the rescue and/or routine inhalations. The processor then determines the probability of the subject having the severe acute respiratory syndrome based on the number of rescue inhalations and the parameters. Further provided is a method for determining the probability of an asthma exacerbation in a subject, which method employs the weighted model.

### TECHNICAL FIELD

This disclosure relates to an inhaler system, and particularly systems and methods for determining a level of acute risk to a subject.

### BACKGROUND

Many respiratory diseases, such as asthma or chronic obstructive pulmonary disease (COPD), are life-long conditions where treatment involves the long-term administration of medicaments to manage the patients' symptoms and to decrease the risks of irreversible changes. There is currently no cure for diseases like asthma and COPD. Treatment takes two forms. First, a maintenance aspect of the treatment is intended to reduce airway inflammation and, consequently, control symptoms in the future. The maintenance therapy is typically provided by inhaled corticosteroids, alone or in combination with long-acting bronchodilators and/or muscarinic antagonists. Secondly, there is also a rescue (or reliever) aspect of the therapy, where patients are given rapid-acting bronchodilators to relieve acute episodes of wheezing, coughing, chest tightness and shortness of breath.

Patients suffering from a respiratory disease, such as asthma or COPD may also experience episodic flare-ups, or exacerbations, in their respiratory disease, where symptoms rapidly worsen. In the worst case, exacerbations may be life-threatening.

The ability to more accurately determine a level of acute risk to a subject suffering from a chronic respiratory disease would improve action plans and provide opportunities for pre-emptive treatment.

There is therefore a need in the art for improved methods of determining the level of acute risk to a subject suffering from a chronic respiratory disease.

Patients suffering from a chronic respiratory disease, such as asthma or COPD, may be particularly vulnerable to a severe acute respiratory syndrome. Damage caused by the chronic respiratory disease to the epithelial lining can increase the susceptibility of the subject to contracting a viral respiratory disease, and viral respiratory infections are linked to increased risk of respiratory disease exacerbations, such as asthma and COPD exacerbations.

### SUMMARY OF THE INVENTION

Accordingly, the present disclosure provides a system for determining the level of acute risk to a subject suffering from a chronic respiratory disease. An exemplary system comprises an inhaler configured to deliver a medicament to the subject for treating the respiratory disease. The inhaler comprises a use determination system configured to determine at least one first value of a usage parameter relating to use of the inhaler.

The exemplary system further comprises a processing module configured to receive the at least one first value. The processing module is also configured to receive a second value indicative of whether the subject is also suffering from a viral respiratory infection.

The processing module is configured to determine the level of acute risk based on the at least one first value and the second value.

Assessing the level of acute risk to the subject based on the usage of the inhaler, as determined via the use determination system, and whether the subject is also suffering from a viral respiratory infection may enable a more accurate assessment of the level of acute risk to the subject. In particular, this risk level determination may have improved accuracy relative to the scenario in which the assessment neglects the possibility that the subject is also suffering from a viral respiratory infection.

Basing the level of acute risk to the subject on the at least one first value and the second value may provide a quantitative measure which can be used to justify, for example, hospital admission, and, at certain risk levels, admission of the subject into an intensive care unit.

Moreover, the same metrics can be used to justify discharge of the subject from the hospital or intensive care unit, for example when the level of acute risk is observed to decrease as a result of the inhaler usage data returning to normal levels and/or by the second value indicating that the subject has recovered from, or has shown a sufficient degree of recovery from, the viral respiratory infection.

### BRIEF DESCRIPTION OF THE DRAWINGS

The embodiments will now be described in more detail with reference to the accompanying drawings, which are not intended to be limiting:

FIG. 1 shows a block diagram of an inhaler according to an example;

FIG. 2 shows a graph of flow rate versus time during use of an inhaler according to an example;

FIG. 3 shows a block diagram of a system according to an example;

FIG. 4 shows front and rear views of the exterior of an inhaler according to an example;

FIG. 5 shows an uppermost surface of the top cap of the inhaler shown in FIG. 4;

FIG. 6 schematically depicts pairing the inhaler shown in FIG. 4 with a user device;

FIG. 7 provides a flowchart of a method according to an example;

FIG. 8 shows a front perspective view of an exemplary inhaler;

FIG. 9 shows a cross-sectional interior perspective view of the inhaler shown in FIG. 8;

FIG. 10 provides an exploded perspective view of the example inhaler shown in FIG. 8;

FIG. 11 provides an exploded perspective view of a top cap and electronics module of the inhaler shown in FIG. 8;

FIG. 12 shows a graph of airflow rate through the example inhaler shown in FIG. 8 versus pressure;

FIG. 13 shows a block diagram of a system according to an example;

FIG. 14 shows a system according to another example; and

FIG. 15 shows a flowchart of a method according to an example.

### DETAILED DESCRIPTION

It should be understood that the detailed description and specific examples, while indicating exemplary embodiments of the apparatus, systems and methods, are intended for purposes of illustration only and are not intended to limit the scope of the invention. These and other features, aspects, and advantages of the apparatus, systems and methods of the present invention will become better understood from the following description, appended claims, and accompanying drawings. It should be understood that the Figures are merely schematic and are not drawn to scale. It should also be understood that the same reference numerals are used throughout the figures to indicate the same or similar parts.

Asthma and COPD are chronic inflammatory disease of the airways. They are both characterized by variable and recurring symptoms of airflow obstruction and bronchospasm. The symptoms include episodes of wheezing, coughing, chest tightness and shortness of breath.

The symptoms are managed by avoiding triggers and by the use of medicaments, particularly inhaled medicaments. The medicaments include inhaled corticosteroids (ICSs) and bronchodilators.

Inhaled corticosteroids (ICSs) are steroid hormones used in the long-term control of respiratory disorders. They function by reducing the airway inflammation. Examples include budesonide, beclomethasone (dipropionate), fluticasone (propionate or furoate), mometasone (furoate), ciclesonide and dexamethasone (sodium). Parentheses indicate preferred salt or ester forms. Particular mention should be made of budesonide, beclomethasone and fluticasone, especially budesonide, beclomethasone dipropionate, fluticasone propionate and fluticasone furoate.

Different classes of bronchodilators target different receptors in the airways. Two commonly used classes are β₂-agonists and anticholinergics.

β₂-Adrenergic agonists (or “β₂-agonists”) act upon the β₂-adrenoceptors which induces smooth muscle relaxation, resulting in dilation of the bronchial passages. They tend to be categorised by duration of action. Examples of long-acting β₂-agonists (LABAs) include formoterol (fumarate), salmeterol (xinafoate), indacaterol (maleate), bambuterol (hydrochloride), clenbuterol (hydrochloride), olodaterol (hydrochloride), carmoterol (hydrochloride), tulobuterol (hydrochloride) and vilanterol (triphenylacetate). Examples of short-acting β₂-agonists (SABA) are albuterol (sulfate) and terbutaline (sulfate). Particular mention should be made of formoterol, salmeterol, indacaterol and vilanterol, especially formoterol fumarate, salmeterol xinafoate, indacaterol maleate and vilanterol triphenylacetate.

Typically short-acting bronchodilators provide a rapid relief from acute bronchoconstriction (and are often called “rescue” or “reliever” medicines), whereas long-acting bronchodilators help control and prevent longer-term symptoms. However, some rapid-onset long-acting bronchodilators may be used as rescue medicines, such as formoterol (fumarate). Thus, a rescue medicine provides relief from acute bronchoconstriction. The rescue medicine is taken as-needed/prn (pro re nata). The rescue medicine may also be in the form of a combination product, e.g. ICS-formoterol (fumarate), typically budesonide-formoterol (fumarate) or beclomethasone (dipropionate)-formoterol (fumarate). Thus, the rescue medicine is preferably a SABA or a rapid-acting LABA, more preferably albuterol (sulfate) or formoterol (fumarate), and most preferably albuterol (sulfate).

Anticholinergics (or “antimuscarinics”) block the neurotransmitter acetylcholine by selectively blocking its receptor in nerve cells. On topical application, anticholinergics act predominantly on the M₃ muscarinic receptors located in the airways to produce smooth muscle relaxation, thus producing a bronchodilatory effect. Examples of long-acting muscarinic antagonists (LAMAs) include tiotropium (bromide), oxitropium (bromide), aclidinium (bromide), umeclidinium (bromide), ipratropium (bromide) glycopyrronium (bromide), oxybutynin (hydrochloride or hydrobromide), tolterodine (tartrate), trospium (chloride), solifenacin (succinate), fesoterodine (fumarate) and darifenacin (hydrobromide). Particular mention should be made of tiotropium, aclidinium, umeclidinium and glycopyrronium, especially tiotropium bromide, aclidinium bromide, umeclidinium bromide and glycopyrronium bromide.

A number of approaches have been taken in preparing and formulating these medicaments for delivery by inhalation, such as via a dry powder inhaler (DPI), a pressurized metered dose inhaler (pMDI) or a nebulizer.

According to the GINA (global Initiative for Asthma) Guidelines, a step-wise approach is taken to the treatment of asthma. At step 1, which represents a mild form of asthma, the patient is given an as needed SABA, such as albuterol sulfate. The patient may also be given an as-needed low-dose ICS-formoterol, or a low-dose ICS whenever the SABA is taken. At step 2, a regular low-dose ICS is given alongside the SABA, or an as-needed low-dose ICS-formoterol. At step 3, a LABA is added. At step 4, the doses are increased and at step 5, further add-on treatments are included such as an anticholinergic or a low-dose oral corticosteroid. Thus, the respective steps may be regarded as treatment regimens, which regimens are each configured according to the degree of acute severity of the respiratory disease.

COPD is a leading cause of death worldwide. It is a heterogeneous long-term disease comprising chronic bronchitis, emphysema and also involving the small airways. The pathological changes occurring in patients with COPD are predominantly localised to the airways, lung parenchyma and pulmonary vasculature. Phenotypically, these changes reduce the healthy ability of the lungs to absorb and expel gases.

Bronchitis is characterised by long-term inflammation of the bronchi. Common symptoms may include wheezing, shortness of breath, cough and expectoration of sputum, all of which are highly uncomfortable and detrimental to the patient's quality of life. Emphysema is also related to long-term bronchial inflammation, wherein the inflammatory response results in a breakdown of lung tissue and progressive narrowing of the airways. In time, the lung tissue loses its natural elasticity and becomes enlarged. As such, the efficacy with which gases are exchanged is reduced and respired air is often trapped within the lung. This results in localised hypoxia, and reduces the volume of oxygen being delivered into the patient's bloodstream, per inhalation. Patients therefore experience shortness of breath and instances of breathing difficulty.

Patients living with COPD experience a variety, if not all, of these symptoms on a daily basis. Their severity will be determined by a range of factors but most commonly will be correlated to the progression of the disease. These symptoms, independent of their severity, are indicative of stable COPD and this disease state is maintained and managed through the administration of a variety drugs. The treatments are variable, but often include inhaled bronchodilators, anticholinergic agents, long-acting and short-acting β₂-agonists and corticosteroids. The medicaments are often administered as a single therapy or as combination treatments.

Patients are categorised by the severity of their COPD using categories defined in the GOLD Guidelines (global Initiative for Chronic Obstructive Lung Disease, Inc.). The categories are labelled A-D and the recommended first choice of treatment varies by category. Patient group A are recommended a short-acting muscarinic antagonist (SAMA) pm or a short-acting β₂-aginist (SABA) pm. Patient group B are recommended a long-acting muscarinic antagonist (LAMA) or a long-acting β₂-aginist (LABA). Patient group C are recommended an inhaled corticosteroid (ICS)+a LABA, or a LAMA. Patient group D are recommended an ICS+a LABA and/or a LAMA.

Patients suffering from respiratory diseases like asthma or COPD suffer from periodic exacerbations beyond the baseline day-to-day variations in their condition. An exacerbation is an acute worsening of respiratory symptoms that require additional therapy, i.e. a therapy going beyond their maintenance therapy.

For asthma, the additional therapy for a moderate exacerbation are repeated doses of SABA, oral corticosteroids and/or controlled flow oxygen (the latter of which requires hospitalization). A severe exacerbation adds an anticholinergic (typically ipratropium bromide), nebulized SABA or IV magnesium sulfate.

For COPD, the additional therapy for a moderate exacerbation are repeated doses of SABA, oral corticosteroids and/or antibiotics. A severe exacerbation adds controlled flow oxygen and/or respiratory support (both of which require hospitalization). An exacerbation within the meaning of the present disclosure includes both moderate and severe exacerbations.

Viral respiratory infections are linked to increased risk of respiratory disease exacerbations, such as asthma and COPD exacerbations. They may be caused by a number of infectious agents including rhinoviruses, influenza viruses, respiratory syncytial virus, adenoviruses, metapneumoviruses and coronaviruses.

Rhinoviruses are RNA viruses, sometimes known as human rhinoviruses (HRVs). HRVs A-C are all relevant. Influenza viruses are RNA viruses and include influenzaviruses A-D. However, influenzaviruses A and B, especially A, are of particular relevance for human infection. Respiratory syncytial virus (RSV) is another RNA virus. It is also known as human respiratory syncytial virus or human orthopneumovirus. Adenoviruses are DNA viruses. They cause a range of infections, but most commonly infect the respiratory system. Metapneumoviruses are RNA viruses sometimes known as human metapneumoviruses (HMPVs). Coronaviruses are enveloped viruses with a positive-sense single-stranded RNA genome. They are responsible for severe acute respiratory syndrome (SARS), including coronavirus disease caused by severe acute respiratory syndrome coronavirus 2 (SARS-CoV-2), also known as Covid-19.

Accordingly, the level of acute risk to a subject having a chronic respiratory disease, such as asthma and COPD, increases when the subject contracts a viral respiratory disease.

Moreover, damage caused by the chronic respiratory disease to the epithelial lining can increase the susceptibility of the subject to contracting the viral respiratory disease.

Attempts have been made to assess the level of acute risk to sufferers of chronic respiratory diseases, such as an asthma or COPD, by monitoring various subject-related and environmental factors. Challenges have been encountered concerning which factors should be taken into account, and which neglected. Neglecting factors which only have a minimal or negligible influence on the risk determination may enable determination of the risk more efficiently, for example using less computational resources, such as processing resources, battery power, memory requirements, etc. Of greater importance is the requirement to improve the accuracy with which the level of acute risk to the subject is determined. A more accurate risk determination may facilitate a more effective warning system so that the appropriate clinical intervention may be delivered to the subject. Thus, more accurate assessment of the level of acute risk may have the potential to guide intervention for a subject at acute risk.

Provided is a system comprising an inhaler. The inhaler comprises a use determination system. The use determination system is configured to determine at least one first value of a usage parameter relating to use of the inhaler. A processing module is configured to receive the at least one first value. The processing module is also configured to receive a second value indicative of whether the subject is also suffering from a viral respiratory infection. The processing module then determines the level of acute risk based on the at least one first value and the second value.

The level of acute risk to the subject may be a measure of the likelihood or probability of mortality within a given time period if the current treatment(s) is or are maintained without any additional clinical intervention.

The given time period may be, for example, three weeks or less, such as two weeks, e.g. 10 days.

For example, the level of acute risk may be a measure of the likelihood of death within the given time period of an un-hospitalized subject if the current out-of-hospital treatment(s) is or are maintained.

Alternatively, the level of acute risk may be a measure of the likelihood of death within the given time period for a subject not currently admitted into an intensive care unit (ICU) if the subject remains out of the ICU and the current out-of-ICU treatment(s) is or are maintained.

The inhaler may, for example, comprise a medicament reservoir containing the medicament.

Whilst not essential in the context of the present disclosure, the system may comprise at least one further inhaler. The at least one further inhaler may be configured to deliver one or more further medicaments to the subject. This would be the same subject to whom the medicament is administered via the inhaler. One or more (or each) of the at least one further inhaler may, for example, comprise a respective further medicament reservoir containing the further medicament.

The medicament and the further medicament may be the same as or different from each other, but usually they will be different from each other.

In a non-limiting example, the medicament is a rescue medicament for use by the subject as needed, and the further medicament is a maintenance medicament which is used by the subject according to a predetermined treatment regimen.

The rescue medicament is as defined hereinabove and is typically a SABA or a rapid-onset LABA, such as formoterol (fumarate). The rescue medicament may also be in the form of a combination product, e.g. ICS-formoterol (fumarate), typically budesonide-formoterol (fumarate) or beclomethasone (dipropionate)-formoterol (fumarate). Such an approach is termed “MART” (maintenance and rescue therapy).

In a non-limiting example, the medicament is selected from albuterol (sulfate), budesonide, beclomethasone (dipropionate), fluticasone (propionate or furoate), formoterol (fumarate), salmeterol (xinafoate), indacaterol (maleate), vilanterol (triphenylacetate), tiotropium (bromide), aclidinium (bromide), umeclidinium (bromide), glycopyrronium (bromide), salmeterol (xinafoate) combined with fluticasone (propionate or furoate), beclomethasone (dipropionate) combined with albuterol (sulfate), and budesonide combined with formoterol (fumarate).

More generally, the medicament, the further medicament, and any other medicaments included in inhalers of the system, may comprise any suitable active pharmaceutical ingredient. Thus, any class of medication for treating the chronic respiratory disease may be delivered by, in other words housed within, the inhaler(s) included in the system.

At least one, e.g. each, inhaler included in the system comprises a use determination system. The use determination system is configured to determine at least one first value of a usage parameter relating to use of the respective inhaler. The usage parameter may, for instance, comprise a use of, such as an inhalation of the medicament performed by the subject using, the respective inhaler. Alternatively or additionally, the usage parameter may comprise a parameter relating to airflow during inhalation of the medicament performed by the subject.

The use determination system may, for example, comprise a sensor for detecting inhalation of the respective medicament performed by the subject and/or a mechanical switch configured to be actuated prior to, during, or after use of the respective inhaler. In this way, the use determination system enables recording of each use, or attempted use, of the inhaler.

Such a sensor may, for example, comprise a pressure sensor, such as an absolute or differential pressure senor.

Determining usage of the inhaler via the use determination system may represent data which is pertinent to the determination of the level of acute risk to the subject. When, for example, the system comprises a rescue inhaler, the number of rescue inhalations can represent a diagnostic factor in determining the level of acute risk to the subject, since the subject may use the rescue inhaler more as their condition deteriorates, e.g. as an exacerbation approaches.

The number of maintenance inhalations using a maintenance inhaler may alternatively or additionally represent useful information for determining the level of acute risk, since fewer maintenance inhalations (indicative of poorer compliance with a maintenance medication treatment regimen) may result in increased risk to the subject, e.g. an increased risk of an exacerbation.

Thus, in an example, a change in the number of rescue inhalations using the rescue inhaler (e.g. an increase or decrease relative to a baseline period for the subject in question) and/or a change in the number of inhalations using the maintenance inhaler (indicative of compliance with or lower adherence to a treatment regimen) may, together with the second value indicative of whether the subject is also suffering from a viral respiratory infection, provide a relatively accurate estimate of the level of acute risk to the subject.

Alternatively or additionally, the usage parameter comprises a parameter relating to airflow during inhalation of the medicament performed by the subject.

To this end, the use determination system may, for example, comprise a sensor for sensing the parameter. In this example, the sensor for sensing the parameter may be the same as or different from the above-described sensor for determining a use of the inhaler.

The parameter relating to airflow during the inhalation(s) may provide an indicator of the level of acute risk, e.g. including the likelihood of an impending exacerbation, since the parameter may act as a proxy for the lung function and/or lung health of the subject.

Thus, in a further example, a change in the number of rescue inhalations using the rescue inhaler (e.g. an increase or decrease relative to a baseline period for the subject in question), a change in the number of inhalations using the maintenance inhaler (indicative of compliance with or lower adherence to a treatment regimen), and/or inhalation parameters indicating a change in lung function may, together with the second value, provide a relatively accurate estimate of the level of acute risk to the subject.

In this example, increased usage of a rescue inhaler, decreased use of a maintenance inhaler relative to the number of uses required by the associated treatment regimen, and/or changes in a parameter relating to airflow during use of the inhaler indicative of deteriorating lung function may, combined with a positive diagnosis of the viral respiratory infection, result in a level of acute risk being determined which warrants additional clinical intervention. Such an intervention may involve admission of the subject into a different healthcare setting, such as a hospital or ICU.

Any suitable parameter relating to airflow can be considered. In a non-limiting example, the parameter is at least one of a peak inhalation flow, an inhalation volume, a time to peak inhalation flow, and an inhalation duration.

In certain examples, the use determination system employs the sensor in combination with the mechanical switch in order to determine the parameter relating to airflow during a use of the inhaler by the subject.

The inhaler may, for instance, comprise a mouthpiece through which the user performs the inhalation, and a mouthpiece cover. In such an example, the mechanical switch may be configured to be actuated when the mouthpiece cover is moved to expose the mouthpiece.

More generally, the system also comprises a processing module which receives the at least one first value. The processing module is also configured to receive a second value indicative of whether the subject is also suffering from a viral respiratory infection.

In an embodiment, the second value is a value obtained from a virus detection method for detecting the virus of the respiratory viral infection in a sample from the subject.

In an embodiment, the viral respiratory infection is at least one selected from a viral respiratory infection caused by a rhinovirus, an influenza virus, a respiratory syncytial virus (RSV), an adenovirus, a metapneumovirus, and a coronavirus.

In a non-limiting example, the viral respiratory infection is severe acute respiratory syndrome (SARS), such as coronavirus disease caused by severe acute respiratory syndrome coronavirus 2 (SARS-CoV-2), also known as Covid-19.

The second value may be, for instance, a positive or a negative result from the virus detection method. In this example, the positive or negative result may be determined according to whether or not an analyte is detectable, in other words whether or not the analyte concentration exceeds a detection limit of the method.

In a non-limiting example, the virus detection method is an assay configured for selective binding of the virus. This is often called an antigen test. Alternatively or additionally, the virus detection method is a nucleic acid detection method for detecting at least part of the viral genome of the virus. This method uses the well-known technique of polymerase chain reaction (PCR) to amplify a small, well-defined segment of DNA. Reverse transcription polymerase chain reaction (RT-PCR) is applied for RNA viruses (including rhinovirus, influenza virus, respiratory syncytial virus, metapneumovirus and coronavirus).

As an alternative to or in addition to the positive/negative result, the second value can comprise a likelihood or probability that the subject is suffering from the viral respiratory disease. Such a likelihood may, for example, be based on the degree to which the subject's symptoms correspond to a predetermined set of symptoms established for the viral respiratory infection. For example, for Covid-19, the subject may enter symptoms such as a high temperature, a new, continuous cough and/or a loss or change to sense of smell or taste.

The second value may be inputted in any suitable manner. The system may, for example, comprise a user interface which is configured to enable inputting of the second value, e.g. by the subject, healthcare provider, etc. That is, the symptoms to be entered may be prompted by a menu provided to the subject from which relevant symptoms may be selected.

The processing module then determines the level of acute risk based on the at least one first value and the second value.

The processing module may include a general purpose processor, a special purpose processor, a DSP, a microcontroller, an integrated circuit, and/or the like that may be configured using hardware and/or software to perform the functions described herein for the processing module. The processing module may be included partially or entirely in the inhaler, a user device, and/or a server.

The processing module may include a power supply, memory, and/or a battery.

The processing module may, for example, be configured to determine the level of acute risk to the subject using a suitable model.

The model used by the processing module for this purpose may be a linear model or may be a non-linear model.

In a particular example, the model is a machine learning model. A supervised model, such as a supervised machine learning model, may, for example, be used.

In a non-limiting example, the model is constructed using a decision trees technique. Other suitable techniques, such as building a neural network or a deep learning model may also be contemplated.

Such an exemplary model may be constructed using, as a training set, the clinical outcomes from a first population of subjects who have been prescribed an inhaler having the above-described use determination system for their chronic respiratory disease, e.g. asthma or COPD, and who have been diagnosed with a viral respiratory infection, such as Covid-19.

The model may, for instance, also use the clinical outcomes from a second population of subjects who have been prescribed an inhaler having the above-described use determination system for their chronic respiratory disease, e.g. asthma or COPD, and who have not been diagnosed with the viral respiratory infection.

The first population may, for example, be divided according to whether or not existing treatment(s) was or were maintained without any additional clinical intervention. In this way, the model may enable estimation of the level of acute risk to the subject if the current treatment(s) is or are continued unchanged based on the at least one first value and the second value.

In an embodiment, the processing module is configured to control a user interface, e.g. the above-described user interface used to enter the second value and/or a further user interface, to issue a notification based on the level of acute risk to the subject.

In a non-limiting example, the user interface may be at least partly defined by a first user interface of a user device. The user device may, for example, be at least one selected from a personal computer, a tablet computer, and a smart phone.

In a non-limiting example, the processing module is at least partly included in a first processing module included in the user device. In other non-limiting examples, the processing module is not included in a user device. The processing module (or at least part of the processing module) may, for example, be provided in a server, e.g. a remote server. For example, the processing module may be implemented on any combination of the inhaler, the user device, and/or a remote server. As such, any combination of the functions or processing described with reference to the processing module may be performed by a processing module residing on the inhaler, the user device, and/or a server. For instance, the use determination system residing on the inhaler may capture usage information at the inhaler (e.g. such as a use or manipulation of the inhaler by the user (such as the opening of a mouthpiece cover and/or the actuation of a switch) and/or the parameter relating to airflow during a use of the inhaler), while the processing module residing on any combination of the inhaler, the user device, and/or server may determine inhalation parameters based on the parameter relating to airflow during a use of the inhaler and/or determine notifications associated with the inhalation parameters.

Further provided is a method for determining a level of acute risk to a subject suffering from a chronic respiratory disease, the method comprising: receiving at least one first value of a usage parameter relating to use of an inhaler from a use determination system included in an inhaler configured to deliver a medicament to the subject for treating the respiratory disease; receiving a second value indicative of whether the subject is also suffering from a viral respiratory infection; and determining the level of acute risk based on the at least one first value and the second value.

A computer program is also provided, which computer program comprises computer program code which is adapted, when the computer program is run on a computer, to implement the method. In an example, the computer code may reside partially or entirely on a user device (e.g. as a mobile application residing on the user device).

The embodiments described herein for the system are applicable to the method and the computer program. Moreover, the embodiments described for the method and computer program are applicable to the system.

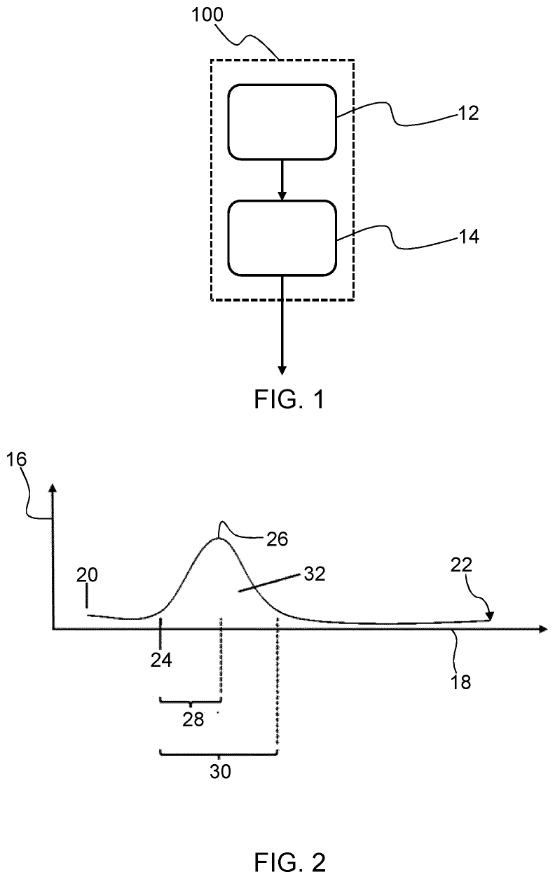

FIG. 1 shows a block diagram of an inhaler 100 according to a non-limiting example. The inhaler 100 comprises a use determination system 12 which determines the at least one first value relating to use of the inhaler 100.

The at least one first value may be communicated from the inhaler 100 to the processing module (not visible in FIG. 1) in any suitable manner.

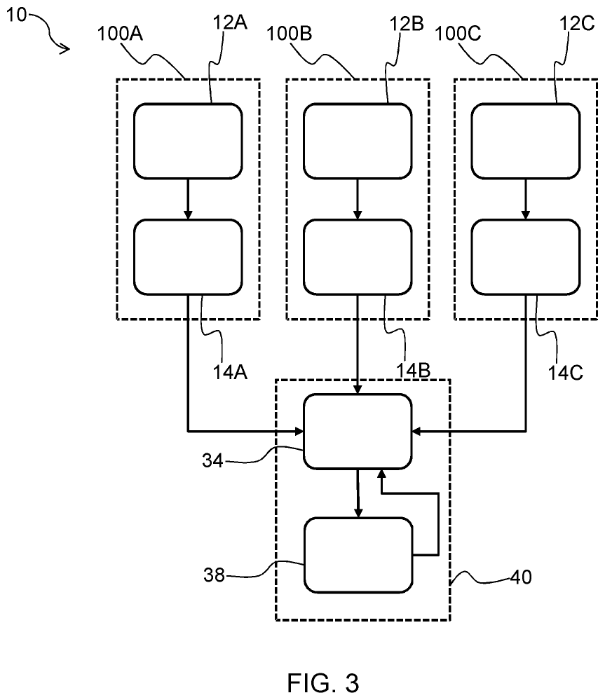

In the non-limiting example shown in FIGS. 1 and 3, the at least one first value is received by a transmission module 14, as represented in FIG. 1 by the arrow between the block representing the use determination system 12 and the block representing the transmission module 14. The transmission module 14 encrypts data based on the at least one first value, and transmits the encrypted data, as represented in FIG. 1 by the arrow pointing away from the transmission module 14 block. The transmission of the encrypted data by the transmission module 14 may, for example, be wireless.

The use determination system 12 may include one or more components used to determine the at least one first value. For example, the use determination system 12 may, for instance, comprise a mechanical switch configured to be actuated prior to, during, or after use of the respective inhaler.

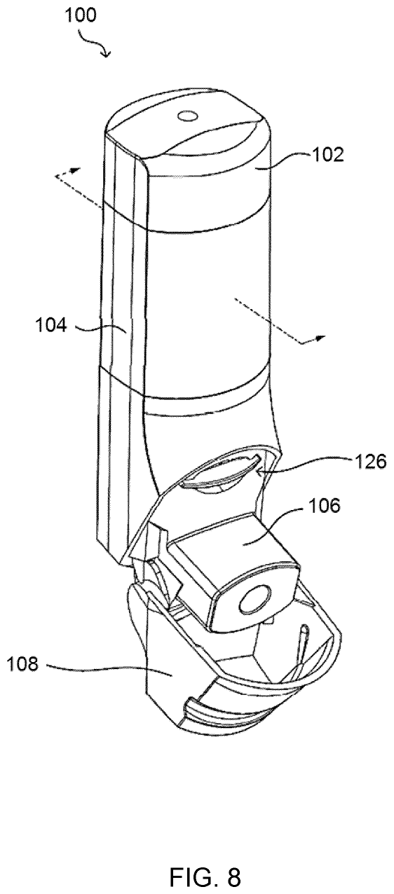

In a non-limiting example, the inhaler 100 comprises a medicament reservoir (not visible in FIG. 1), and a dose metering assembly (not visible in FIG. 1) configured to meter a dose of the rescue medicament from the reservoir. The use determination system 12 may be configured to register the metering of the dose by the dose metering assembly, each metering being thereby indicative of a use (or attempted use) of the inhaler 100. One non-limiting example of the dose metering assembly will be explained in greater detail with reference to FIGS. 8-11.

Alternatively or additionally, the use determination system 12 may register each inhalation in different manners and/or based on additional or alternative feedback. For example, the use determination system 12 is configured to register a use or attempted use of the inhaler by the subject when the feedback from a suitable sensor (not visible in FIG. 1) indicates that an inhalation by the subject has occurred, for example when a pressure change measurement or flow rate exceeds a predefined threshold associated with an inhalation, and/or when a duration of a pressure change above a threshold exceeds a predefined time threshold associated with a low duration inhalation or a good duration inhalation.

A sensor, such as a pressure sensor, may, for example, be included in the use determination system 12 in order to determine the parameter relating to airflow during use, e.g. each use, of the inhaler. When a pressure sensor is included in the use determination system 12, the pressure sensor may, for instance, be used to confirm that, or assess the degree to which, a dose metered via the dose metering assembly is inhaled by the subject, as will be described in greater detail with reference to FIGS. 2 and 8-12.

More generally, the use determination system 12 may comprise a sensor for detecting a parameter relating to airflow during inhalation of the respective medicament performed by the subject. In other words, the usage parameter comprises a parameter relating to airflow during inhalation of the medicament.

The parameter may comprise, for example, at least one of a peak inhalation flow, an inhalation volume, a time to peak inhalation flow, and an inhalation duration. In such examples, the at least one first value may comprise a numerical value for the peak inhalation flow, the inhalation volume, the time to peak inhalation flow, and/or the inhalation duration.

A pressure sensor may be particularly suitable for measuring the parameter, since the airflow during inhalation by the subject may be monitored by measuring the associated pressure changes. As will be explained in greater detail with reference to FIGS. 8-12, the pressure sensor may be located within or placed in fluid communication with a flow pathway through which air and the medicament is drawn by the subject during inhalation. Alternative ways of measuring the parameter, such as via a suitable flow sensor, can also be used.

An inhalation may be associated with a decrease in the pressure in the airflow channel of the inhaler relative to when no inhalation is taking place. The point at which the pressure change is at its greatest may correspond to the peak inhalation flow. The pressure sensor may detect this point in the inhalation.

The pressure change associated with an inhalation may alternatively or additionally be used to determine an inhalation volume. This may be achieved by, for example, using the pressure change during the inhalation measured by the pressure sensor to first determine the flow rate over the time of the inhalation, from which the total inhaled volume may be derived.

The pressure change associated with an inhalation may alternatively or additionally be used to determine an inhalation duration. The time may be recorded, for example, from the first decrease in pressure measured by the pressure sensor, coinciding with the start of the inhalation, to the pressure returning to a pressure corresponding to no inhalation taking place.

The inhalation parameter may alternatively or additionally include the time to peak inhalation flow. This time to peak inhalation flow parameter may be recorded, for example, from the first decrease in pressure measured by the pressure sensor, coinciding with the start of the inhalation, to the pressure reaching a minimum value corresponding to peak flow.

FIG. 2 shows a graph of flow rate 16 versus time 18 during use of an inhaler 100 according to a non-limiting example. The use determination system 12 in this example comprises a mechanically operated switch in the form of a switch which is actuated when a mouthpiece cover of the inhaler 100 is opened. The mouthpiece cover is opened at point 20 on the graph. In this example, the use determination system 12 further comprises a pressure sensor.

When the mouthpiece cover is opened, the use determination system 12 is woken out of an energy-saving sleep mode, and a new inhalation event is registered. The inhalation event is also assigned an open time corresponding to how much time, for example in milliseconds, elapses since the inhaler 100 wakes from the sleep mode. Point 22 corresponds to the cap closing or 60 seconds having elapsed since point 20. At point 22, detection ceases.

Once the mouthpiece cover is open, the use determination system 12 looks for a change in the air pressure, as detected using the pressure sensor. The start of the air pressure change is registered as the inhale event time 24. The point at which the air pressure change is greatest corresponds to the peak inhalation flow 26. The use determination system 12 records the peak inhalation flow 26 as a flow of air, measured in units of 100 mL per minute, which flow of air is transformed from the air pressure change. Thus, in this example, the at least one first value includes a numerical value of the peak inhalation flow in units of 100 mL per minute.

The time to peak inhalation flow 28 corresponds to the time taken in milliseconds for the peak inhalation flow 26 to be reached. The inhalation duration 30 corresponds to the duration of the entire inhalation in milliseconds. The area under the graph 32 corresponds to the inhalation volume in milliliters.

In a non-limiting example, the inhaler 100 is configured such that, for a normal inhalation, the medicament is dispensed approximately 0.5 seconds following the start of the inhalation. A subject's inhalation only reaching peak inhalation flow after the 0.5 seconds have elapsed, such as after approximately 1.5 seconds, may be partially indicative of the subject having difficulty in controlling their respiratory disease. Such a time to reach peak inhalation flow may, for example, be indicative of a heightened level of acute risk to the subject, e.g. the subject facing an impending exacerbation.

More generally, the use determination system 12 may employ respective sensors (e.g. respective pressure sensors) for registering an inhalation/use of the inhaler and detecting the inhalation parameter, or a common sensor (e.g. a common pressure sensor) which is configured to fulfill both inhalation/use registering and inhalation parameter detecting functions.

Any suitable sensor may be included in the use determination system 12, such as one or more pressure sensors, temperature sensors, humidity sensors, orientation sensors, acoustic sensors, and/or optical sensors. The pressure sensor(s) may include a barometric pressure sensor (e.g. an atmospheric pressure sensor), a differential pressure sensor, an absolute pressure sensor, and/or the like. The sensors may employ microelectromechanical systems (MEMS) and/or nanoelectromechanical systems (NEMS) technology.

In a non-limiting example, the use determination system 12 comprises a differential pressure sensor. The differential pressure sensor may, for instance, comprise a dual port type sensor for measuring a pressure difference across a section of the air passage through which the subject inhales. A single port gauge type sensor may alternatively be used. The latter operates by measuring the difference in pressure in the air passage during inhalation and when there is no flow. The difference in the readings corresponds to the pressure drop associated with inhalation.

In another non-limiting example, the use determination system 12 includes an acoustic sensor. The acoustic sensor in this example is configured to sense a noise generated when the subject inhales through the respective inhaler 100. The acoustic sensor may include, for example, a microphone. The respective inhaler 100 may, for instance, comprise a capsule which is arranged to spin when the subject inhales though the device; the spinning of the capsule generating the noise for detection by the acoustic sensor. The spinning of the capsule may thus provide a suitably interpretable noise, e.g. rattle, for deriving the at least one first value, e.g. use and/or inhalation parameter data.

An algorithm may, for example, be used to interpret the acoustic data in order to determine use data and/or the parameter relating to airflow during the inhalation. For instance, an algorithm as described by P. Colthorpe et al., “Adding Electronics to the Breezhaler: Satisfying the Needs of Patients and Regulators”, Respiratory Drug Delivery 2018, 1, 71-80 may be used. Once the generated sound is detected, the algorithm may process the raw acoustic data to generate the use and/or inhalation parameter data.

FIG. 3 shows a block diagram of a system 10 according to a non-limiting example. The system 10 may, for example, be alternatively termed “an inhaler assembly”.

As shown in FIG. 3, the system 10 comprises a first inhaler 100A comprising a first use determination system 12A, and a first transmission module 14A. This exemplary system 10 further comprises a second inhaler 100B comprising a second use determination system 12B, and a second transmission module 14B. The first inhaler 100A delivers a first medicament, and the second inhaler 100B delivers a second medicament which is different from the first medicament.

The exemplary system 10 depicted in FIG. 3 further comprises a third inhaler 100C comprising a third use determination system 12C, and a third transmission module 14C. The third inhaler 100C delivers a third medicament which is different from the first and second medicaments. In other examples, no third inhaler 100C is included in the system 10, or a fourth, fifth, etc. inhaler (not visible) is included in addition to the first inhaler 100A, the second inhaler 100B, and the third inhaler 100C. Alternatively or additionally, the system 10 includes a plurality of first inhalers 100A, a plurality of second inhalers 100B, and so on.

As shown in FIG. 3, the processing module 34 is configured to receive the respective encrypted data transmitted from one or more, e.g. each, of the transmission modules 14A, 14B, 14C, as represented in FIG. 3 by the arrows between each of the blocks corresponding to the transmission modules 14A, 14B, 14C and the block corresponding to the processing module 34. The first, second, and/or third encrypted data may be transmitted wirelessly to the processing module 34, as previously described. The processing module 34 may thus comprise a suitable receiver or transceiver for receiving the encrypted data. The receiver or transceiver of processing module 34 may be configured to implement the same communication protocols as transmission modules 14A, 14B, 14C and may thus include similar communication hardware and software as transmission modules 14A, 14B, 14C as described herein.

Bluetooth communications between one or more, e.g. each, of the inhaler(s) 100A, 100B, 100C and the processing module 34 may enable relatively rapid transmission of the data from the former to the latter. For example, the longest time taken for the data to be transmitted to the processing module 34 may be around 3 minutes when the respective inhaler 100A, 100B, 100C is in Bluetooth range of the processing module 34.

The processing module 34 may comprise a suitable processor and memory configured to perform the functions/methods described herein. For example, the processor may be a general purpose processor programmed with computer executable instructions for implementing the functions of the processing module 34. The processor may be implemented using a microprocessor or microcontroller configured to perform the functions of the processing module 34. The processor may be implemented using an embedded processor or digital signal processor configured to perform the functions of the processing module 34. In an example, the processor may be implemented on a smartphone or other consumer electronic device that is capable of communicating with transmission modules 14A, 14B, 14C and performing the functions of the processing module 34 as described herein. For example, the processing module 34 may be implemented on a smart phone or consumer electronic device that includes an application (e.g. app) that causes the processor of the smartphone or other consumer electronic device to perform the functions of the processing module 34 as described herein.

The system 10 further comprises a user interface 38. The user interface 38 may be configured to enable inputting of the second value indicative of whether the subject is also suffering from a viral respiratory infection and/or to issue a notification based on the level of risk determined by the processing module 34. The subject and/or healthcare provider may then act according to the notified level of risk to the subject.

The arrow pointing from the block representing the processing module 34 to the block representing the user interface 38 is intended to represent the control signal(s) which cause or causes the user interface 38 to issue the notification. In this respect, the user interface 38 may comprise any suitable display, screen, for example touchscreen, etc. which is capable of displaying the notification. Alternatively or additionally, the notification may be provided by the user interface 38 via an audio message. In such an example, the user interface 38 comprises a suitable loudspeaker for delivering the audio message. Numerous ways of communicating the respective usage information can be used.

In the non-limiting example shown in FIG. 3, the arrow pointing from the block representing the user interface 38 to the block representing the processing module 34 is intended to represent the processing module 34 receiving the second value inputted via the user interface 38.

In other examples, respective, i.e. different, user interfaces are used for issuing the notification and inputting the second value.

Whilst the transmission modules 14A, 14B, 14C are each shown in FIG. 3 as transmitting (encrypted) data to the processing module 34, this is not intended to exclude the respective inhalers 100A, 100B, 100C, or a component module thereof, receiving data transmitted from the processing module 34.

Whilst not shown in FIG. 3, the processing module 34 may, in some examples, comprise a clock module, with each of the respective inhalers 100A, 100B, 100C having a further clock module. The further clock modules can be synchronized according to the time provided by the clock module. The clock module may, for instance, receive the time of the time zone in which the processing module 34 is situated. This may cause the respective inhalers 100A, 100B, 100C to be synchronized according to the time in which the subject and their respective inhalers 100A, 100B, 100C are located. In such an example, the processing module 34 may be configured to synchronize the further clock modules of the respective inhalers 100A, 100B, 100C.

Moreover, such synchronization may, for instance, provide a point of reference which enables the relative timing of use of the respective inhalers 100A, 100B, 100C to be determined, which may have clinical relevance. For example, such synchronization may permit a correlation to be drawn between failure of the subject to administer a maintenance medicament at regular times and increased rescue inhaler usage during the same period.

In an embodiment, the processing module 34 is at least partly included in a first processing module included in the user device 40. By implementing as much processing as possible of the usage data from the respective inhalers 100A, 100B, 100C in the first processing module of the user device 40, battery life in the respective inhalers 100A, 100B, 100C may be advantageously saved. The user device 40 may be, for example, at least one selected from a personal computer, a tablet computer, and a smart phone.

Alternatively or additionally, the user interface 38 may be at least partly defined by a first user interface of the user device 40. The first user interface of the user device 40 may, for instance, comprise, or be defined by, the touchscreen of a smart phone 40.

In other non-limiting examples, the processing module is not included in a user device. The processing module 34 (or at least part of the processing module 34) may, for example, be provided in a server, e.g. a remote server.

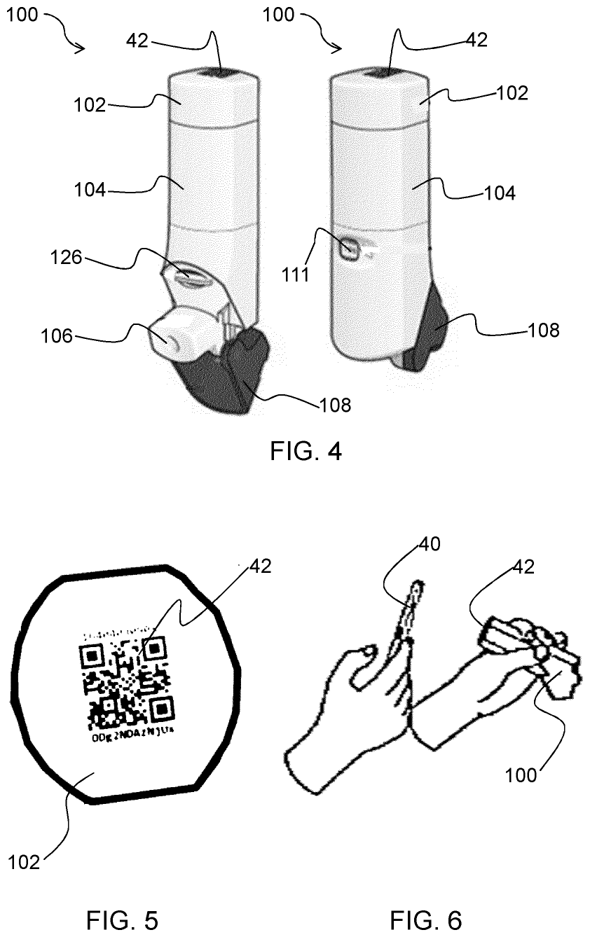

FIG. 4 shows front and rear views of the exterior of an inhaler 100 according to a non-limiting example. The inhaler 100 comprises a top cap 102, a main housing 104, a mouthpiece 106, a mouthpiece cover 108, and an air vent 126. The mouthpiece cover 108 may be hinged to the main housing 104 so that it may open and close to expose the mouthpiece 106 and the air vent 126. The depicted inhaler 100 also comprises a mechanical dose counter 111, whose dose count may be used to check the number of doses remaining as determined by the processing module (on the basis of the total number of doses contained by the inhaler 100 prior to use and on the uses determined by the use determination system 12).

In the non-limiting example shown in FIG. 4, the inhaler 100 has a barcode 42 printed thereon. The barcode 42 in this example is a quick reference (QR) code printed on the uppermost surface of the top cap 102. The use determination system 12 and/or the transmission module 14 may, for example, be located at least partly within the top cap 102, for example as components of an electronics module (not visible in FIG. 4). The electronics module of the inhaler 100 will be described in greater detail with reference to FIGS. 8 to 11.

The QR code is more clearly visible in FIG. 5, which provides a view from directly above the top cap 102 of the inhaler 100 shown in FIG. 4. The QR code 42 may provide a facile way of pairing the respective inhaler 100 with the processing module 34, in examples in which the user device 40 comprises a suitable optical reader, such as a camera, for reading the QR code. FIG. 6 shows a user pairing the inhaler 100 with the processing module 34 using the camera included in the user device 40, which in this particular example is a smart phone.

In other non-limiting examples, the processing module 34 may be paired with the respective inhaler 100 by, for example, manual entry of an alphanumerical key including the respective identifier via the user interface, e.g. a touchscreen.

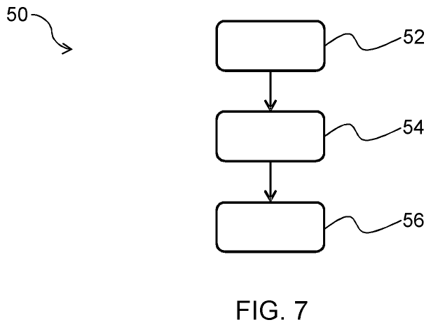

FIG. 7 provides a flowchart of a method 50 according to an example. The method comprises receiving 52 at least one first value of a usage parameter relating to use of an inhaler from a use determination system included in an inhaler configured to deliver a medicament to a subject. The medicament is for treating a chronic respiratory disease. The method 50 further comprises receiving 54 a second value indicative of whether the subject is also suffering from a viral respiratory infection. The level of acute risk is then determined in step 56 based on the at least one first value and the second value.

In an embodiment, the determination 56 utilizes the above-described model.

This method 50 may, for example, be implemented by the processing module 34 of the system 10 described above. In some non-limiting examples, the method 50 is implemented by the processing module 34 residing on a user device, such as a smart phone or tablet.

Also provided is a computer program comprising computer program code which is adapted, when the computer program is run on a computer, to implement any of the above-described methods. In a preferred embodiment, the computer program takes the form of an app, for example an app for a user device 40, such as a mobile device, e.g. tablet computer or a smart phone.

FIGS. 8-11 provide a non-limiting example of an inhaler 100 which may be included in the system 10.

FIG. 8 provides a front perspective view of an inhaler 100 according to a non-limiting example. The inhaler 100 may, for example, be a breath-actuated inhaler. The inhaler 100 may include a top cap 102, a main housing 104, a mouthpiece 106, a mouthpiece cover 108, an electronics module 120, and/or an air vent 126. The mouthpiece cover 108 may be hinged to the main housing 104 so that it may open and close to expose the mouthpiece 106. Although illustrated as a hinged connection, the mouthpiece cover 106 may be connected to the inhaler 100 through other types of connections. Moreover, while the electronics module 120 is illustrated as housed within the top cap 102 at the top of the main housing 104, the electronics module 120 may be integrated and/or housed within the main body 104 of the inhaler 100.

The electronics module 120 may, for instance, include the above-described use determination system 12 and the transmission module 14. For example, the electronics module 120 may include a processor, memory configured to perform the functions of use determination system 12 and/or transmission module 14. The electronics module 120 may include switch(es), sensor(s), slider(s), and/or other instruments or measurement devices configured to determine inhaler usage information as described herein. The electronics module 120 may include a transceiver and/or other communication chips or circuits configured to perform the transmission functions of transmission module 14.

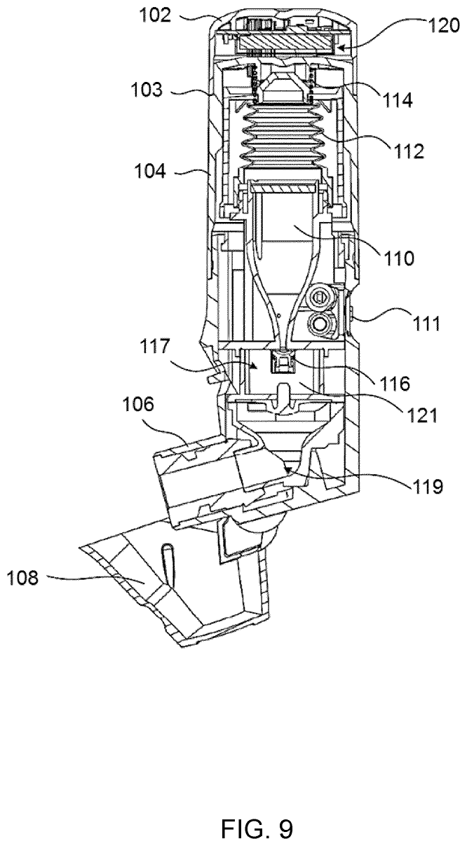

FIG. 9 provides a cross-sectional interior perspective view of the example inhaler 100. Inside the main housing 104, the inhalation device 100 may include a medication reservoir 110 and a dose delivery mechanism. For example, the inhaler 100 may include a medication reservoir 110 (e.g. a hopper), a bellows 112, a bellows spring 114, a yoke (not visible), a dosing cup 116, a dosing chamber 117, a deagglomerator 121, and a flow pathway 119. The medication reservoir 110 may include medication, such as dry powder medication, for delivery to the subject. Although illustrated as a combination of the bellows 112, the bellows spring 114, the yoke, the dosing cup 116, the dosing chamber 117, and the deagglomerator 121, the dose delivery mechanism may include a subset of the components described and/or the inhalation device 100 may include a different dose delivery mechanism (e.g. based on the type of inhalation device, the type of medication, etc.). For instance, in some examples the medication may be included in a blister strip and the dose delivery mechanism, which may include one or more wheels, levers, and/or actuators, is configured to advance the blister strip, open a new blister that includes a dose of medication, and make that dose of medication available to a dosing chamber and/or mouthpiece for inhalation by the user.

When the mouthpiece cover 108 is moved from the closed to the open position, the dose delivery mechanism of the inhaler 100 may prime a dose of medicament. In the illustrated example of FIG. 9, the mouthpiece cover 108 being moved from the closed to the open position may cause the bellows 112 to compress to deliver a dose of medication from the medication reservoir 110 to the dosing cup 116. Thereafter, a subject may inhale through the mouthpiece 106 in an effort to receive the dose of medication.

The airflow generated from the subject's inhalation may cause the deagglomerator 121 to aerosolize the dose of medication by breaking down the agglomerates of the medicament in the dose cup 116. The deagglomerator 121 may be configured to aerosolize the medication when the airflow through the flow pathway 119 meets or exceeds a particular rate, or is within a specific range. When aerosolized, the dose of medication may travel from the dosing cup 116, into the dosing chamber 117, through the flow pathway 119, and out of the mouthpiece 106 to the subject. If the airflow through the flow pathway 119 does not meet or exceed a particular rate, or is not within a specific range, the medication may remain in the dosing cup 116. In the event that the medication in the dosing cup 116 has not been aerosolized by the deagglomerator 121, another dose of medication may not be delivered from the medication reservoir 110 when the mouthpiece cover 108 is subsequently opened. Thus, a single dose of medication may remain in the dosing cup until the dose has been aerosolized by the deagglomerator 121. When a dose of medication is delivered, a dose confirmation may be stored in memory at the inhaler 100 as dose confirmation information.

As the subject inhales through the mouthpiece 106, air may enter the air vent to provide a flow of air for delivery of the medication to the subject. The flow pathway 119 may extend from the dosing chamber 117 to the end of the mouthpiece 106, and include the dosing chamber 117 and the internal portions of the mouthpiece 106. The dosing cup 116 may reside within or adjacent to the dosing chamber 117. Further, the inhaler 100 may include a dose counter 111 that is configured to be initially set to a number of total doses of medication within the medication reservoir 110 and to decrease by one each time the mouthpiece cover 108 is moved from the closed position to the open position.

The top cap 102 may be attached to the main housing 104. For example, the top cap 102 may be attached to the main housing 104 through the use of one or more clips that engage recesses on the main housing 104. The top cap 102 may overlap a portion of the main housing 104 when connected, for example, such that a substantially pneumatic seal exists between the top cap 102 and the main housing 104.

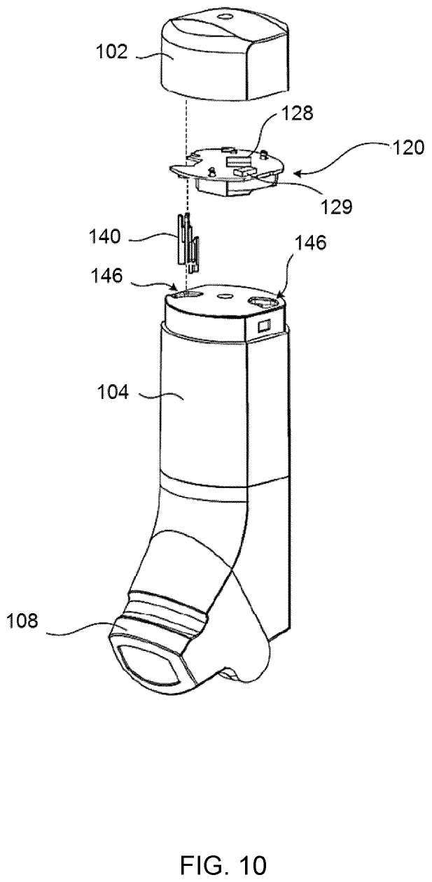

FIG. 10 is an exploded perspective view of the example inhaler 100 with the top cap 102 removed to expose the electronics module 120. As shown in FIG. 10, the top surface of the main housing 104 may include one or more (e.g. two) orifices 146. One of the orifices 146 may be configured to accept a slider 140. For example, when the top cap 102 is attached to the main housing 104, the slider 140 may protrude through the top surface of the main housing 104 via one of the orifices 146.

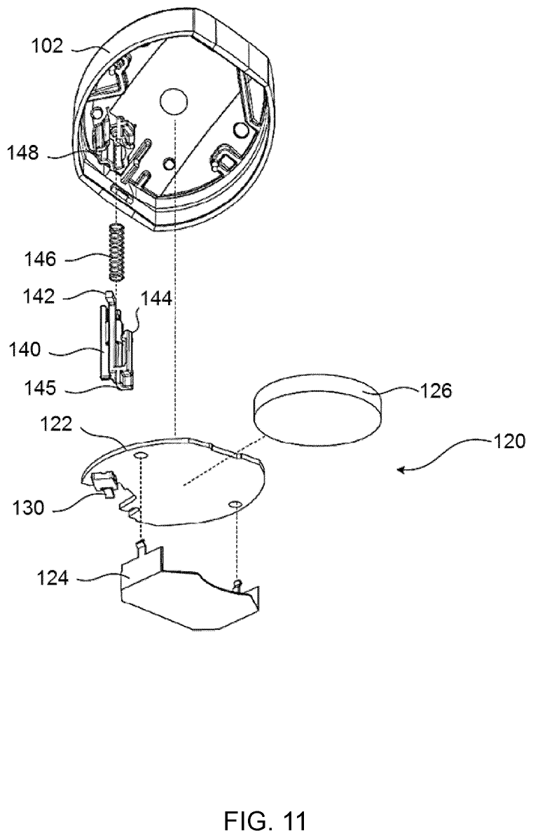

FIG. 11 is an exploded perspective view of the top cap 102 and the electronics module 120 of the example inhaler 100. As shown in FIG. 11, the slider 140 may define an arm 142, a stopper 144, and a distal end 145. The distal end 145 may be a bottom portion of the slider 140. The distal end 145 of the slider 140 may be configured to abut the yoke that resides within the main housing 104 (e.g. when the mouthpiece cover 108 is in the closed or partially open position). The distal end 145 may be configured to abut a top surface of the yoke when the yoke is in any radial orientation. For example, the top surface of the yoke may include a plurality of apertures (not shown), and the distal end 145 of the slider 140 may be configured to abut the top surface of the yoke, for example, whether or not one of the apertures is in alignment with the slider 140.

The top cap 102 may include a slider guide 148 that is configured to receive a slider spring 146 and the slider 140. The slider spring 146 may reside within the slider guide 148. The slider spring 146 may engage an inner surface of the top cap 102, and the slider spring 146 may engage (e.g. abut) an upper portion (e.g. a proximate end) of the slider 140. When the slider 140 is installed within the slider guide 148, the slider spring 146 may be partially compressed between the top of the slider 140 and the inner surface of the top cap 102. For example, the slider spring 146 may be configured such that the distal end 145 of the slider 140 remains in contact with the yoke when the mouthpiece cover 108 is closed. The distal end 145 of the slider 145 may also remain in contact with the yoke while the mouthpiece cover 108 is being opened or closed. The stopper 144 of the slider 140 may engage a stopper of the slider guide 148, for example, such that the slider 140 is retained within the slider guide 148 through the opening and closing of the mouthpiece cover 108, and vice versa. The stopper 144 and the slider guide 148 may be configured to limit the vertical (e.g. axial) travel of the slider 140. This limit may be less than the vertical travel of the yoke. Thus, as the mouthpiece cover 108 is moved to a fully open position, the yoke may continue to move in a vertical direction towards the mouthpiece 106 but the stopper 144 may stop the vertical travel of the slider 140 such that the distal end 145 of the slider 140 may no longer be in contact with the yoke.

More generally, the yoke may be mechanically connected to the mouthpiece cover 108 and configured to move to compress the bellows spring 114 as the mouthpiece cover 108 is opened from the closed position and then release the compressed bellows spring 114 when the mouthpiece cover reaches the fully open position, thereby causing the bellows 112 to deliver the dose from the medication reservoir 110 to the dosing cup 116. The yoke may be in contact with the slider 140 when the mouthpiece cover 108 is in the closed position. The slider 140 may be arranged to be moved by the yoke as the mouthpiece cover 108 is opened from the closed position and separated from the yoke when the mouthpiece cover 108 reaches the fully open position. This arrangement may be regarded as a non-limiting example of the previously described dose metering assembly, since opening the mouthpiece cover 108 causes the metering of the dose of the medicament.

The movement of the slider 140 during the dose metering may cause the slider 140 to engage and actuate a switch 130. The switch 130 may trigger the electronics module 120 to register the dose metering. The slider 140 and switch 130 together with the electronics module 120 may thus be regarded as being included in the use determination system 12 described above. The slider 140 may be regarded in this example as the means by which the use determination system 12 is configured to register the metering of the dose by the dose metering assembly, each metering being thereby indicative of the inhalation performed by the subject using the inhaler 100.

Actuation of the switch 130 by the slider 140 may also, for example, cause the electronics module 120 to transition from the first power state to a second power state, and to sense an inhalation by the subject from the mouthpiece 106.

The electronics module 120 may include a printed circuit board (PCB) assembly 122, a switch 130, a power supply (e.g. a battery 126), and/or a battery holder 124. The PCB assembly 122 may include surface mounted components, such as a sensor system 128, a wireless communication circuit 129, the switch 130, and or one or more indicators (not shown), such as one or more light emitting diodes (LEDs). The electronics module 120 may include a controller (e.g. a processor) and/or memory. The controller and/or memory may be physically distinct components of the PCB 122. Alternatively, the controller and memory may be part of another chipset mounted on the PCB 122, for example, the wireless communication circuit 129 may include the controller and/or memory for the electronics module 120. The controller of the electronics module 120 may include a microcontroller, a programmable logic device (PLD), a microprocessor, an application specific integrated circuit (ASIC), a field programmable gate array (FPGA), or any suitable processing device or control circuit.

The controller may access information from, and store data in the memory. The memory may include any type of suitable memory, such as non-removable memory and/or removable memory. The non-removable memory may include random-access memory (RAM), read-only memory (ROM), a hard disk, or any other type of memory storage device. The removable memory may include a subscriber identity module (SIM) card, a memory stick, a secure digital (SD) memory card, and the like. The memory may be internal to the controller. The controller may also access data from, and store data in, memory that is not physically located within the electronics module 120, such as on a server or a smart phone.

The sensor system 128 may include one or more sensors. The sensor system 128 may be, for example, included in the use determination system 12 described above. The sensor system 128 may include one or more sensors, for example, of different types, such as, but not limited to one or more pressure sensors, temperature sensors, humidity sensors, orientation sensors, acoustic sensors, and/or optical sensors. The one or more pressure sensors may include a barometric pressure sensor (e.g. an atmospheric pressure sensor), a differential pressure sensor, an absolute pressure sensor, and/or the like. The sensors may employ microelectromechanical systems (MEMS) and/or nanoelectromechanical systems (NEMS) technology. The sensor system 128 may be configured to provide an instantaneous reading (e.g. pressure reading) to the controller of the electronics module 120 and/or aggregated readings (e.g. pressure readings) over time. As illustrated in FIGS. 9 and 10, the sensor system 128 may reside outside the flow pathway 119 of the inhaler 100, but may be pneumatically coupled to the flow pathway 119.

The controller of the electronics module 120 may receive signals corresponding to measurements from the sensor system 128. The controller may calculate or determine one or more airflow metrics using the signals received from the sensor system 128. The airflow metrics may be indicative of a profile of airflow through the flow pathway 119 of the inhaler 100. For example, if the sensor system 128 records a change in pressure of 0.3 kilopascals (kPa), the electronics module 120 may determine that the change corresponds to an airflow rate of approximately 45 liters per minute (Lpm) through the flow pathway 119.

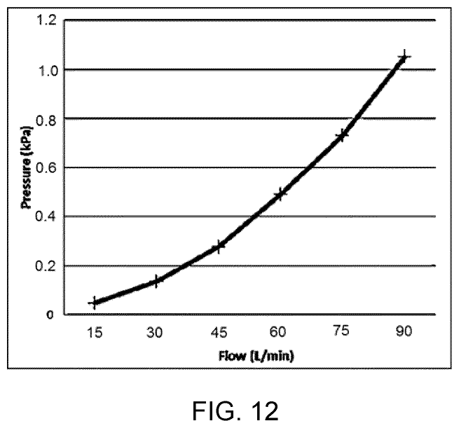

FIG. 12 shows a graph of airflow rates versus pressure. The airflow rates and profile shown in FIG. 12 are merely examples and the determined rates may depend on the size, shape, and design of the inhalation device 100 and its components.

The processing module 34 may generate personalized data in real-time by comparing signals received from the sensor system 128 and/or the determined airflow metrics to one or more thresholds or ranges, for example, as part of an assessment of how the inhaler 100 is being used and/or whether the use is likely to result in the delivery of a full dose of medication. For example, where the determined airflow metric corresponds to an inhalation with an airflow rate below a particular threshold, the processing module 34 may determine that there has been no inhalation or an insufficient inhalation from the mouthpiece 106 of the inhaler 100. If the determined airflow metric corresponds to an inhalation with an airflow rate above a particular threshold, the processing module 34 may determine that there has been an excessive inhalation from the mouthpiece 106. If the determined airflow metric corresponds to an inhalation with an airflow rate within a particular range, the processing module 34 may determine that the inhalation is “good”, or likely to result in a full dose of medication being delivered.

The pressure measurement readings and/or the computed airflow metrics may be indicative of the quality or strength of inhalation from the inhaler 100. For example, when compared to a particular threshold or range of values, the readings and/or metrics may be used to categorize the inhalation as a certain type of event, such as a good inhalation event, a low inhalation event, a no inhalation event, or an excessive inhalation event. The categorization of the inhalation may be usage parameters stored as personalized data of the subject.

The no or low inhalation event may be associated with pressure measurement readings and/or airflow metrics below a particular threshold, such as an airflow rate less than or equal to 30 Lpm. The no inhalation event may occur when a subject does not inhale from the mouthpiece 106 after opening the mouthpiece cover 108 and during the measurement cycle. The no or low inhalation event may also occur when the subject's inspiratory effort is insufficient to ensure proper delivery of the medication via the flow pathway 119, such as when the inspiratory effort generates insufficient airflow to activate the deagglomerator 121 and, thus, aerosolize the medication in the dosing cup 116.

A fair inhalation event may be associated with pressure measurement readings and/or airflow metrics within a particular range, such as an airflow rate greater than 30 Lpm and less than or equal to 45 Lpm. The fair inhalation event may occur when the subject inhales from the mouthpiece 106 after opening the mouthpiece cover 108 and the subject's inspiratory effort causes at least a partial dose of the medication to be delivered via the flow pathway 119. That is, the inhalation may be sufficient to activate the deagglomerator 121 such that at least a portion of the medication is aerosolized from the dosing cup 116.

The good inhalation event may be associated with pressure measurement readings and/or airflow metrics above the low inhalation event, such as an airflow rate which is greater than 45 Lpm and less than or equal to 200 Lpm. The good inhalation event may occur when the subject inhales from the mouthpiece 106 after opening the mouthpiece cover 108 and the subject's inspiratory effort is sufficient to ensure proper delivery of the medication via the flow pathway 119, such as when the inspiratory effort generates sufficient airflow to activate the deagglomerator 121 and aerosolize a full dose of medication in the dosing cup 116.

The excessive inhalation event may be associated with pressure measurement readings and/or airflow metrics above the good inhalation event, such as an airflow rate above 200 Lpm. The excessive inhalation event may occur when the subject's inspiratory effort exceeds the normal operational parameters of the inhaler 100. The excessive inhalation event may also occur if the device 100 is not properly positioned or held during use, even if the subject's inspiratory effort is within a normal range. For example, the computed airflow rate may exceed 200 Lpm if the air vent is blocked or obstructed (e.g. by a finger or thumb) while the subject is inhaling from the mouthpiece 106.

Any suitable thresholds or ranges may be used to categorize a particular event. Some or all of the events may be used. For example, the no inhalation event may be associated with an airflow rate which is less than or equal to 45 Lpm and the good inhalation event may be associated with an airflow rate which is greater than 45 Lpm and less than or equal to 200 Lpm. As such, the low or fair inhalation event may not be used at all in some cases.

The pressure measurement readings and/or the computed airflow metrics may also be indicative of the direction of flow through the flow pathway 119 of the inhaler 100. For example, if the pressure measurement readings reflect a negative change in pressure, the readings may be indicative of air flowing out of the mouthpiece 106 via the flow pathway 119. If the pressure measurement readings reflect a positive change in pressure, the readings may be indicative of air flowing into the mouthpiece 106 via the flow pathway 119. Accordingly, the pressure measurement readings and/or airflow metrics may be used to determine whether a subject is exhaling into the mouthpiece 106, which may signal that the subject is not using the device 100 properly.

The inhaler 100 may include a spirometer or similarly operating device to enable measurement of lung function metrics. For example, the inhaler 100 may perform measurements to obtain metrics related to a subject's lung capacity. The spirometer or similarly operating device may measure the volume of air inhaled and/or exhaled by the subject. The spirometer or similarly operating device may use pressure transducers, ultrasound, or a water gauge to detect the changes in the volume of air inhaled and/or exhaled.

The personalized data collected from, or calculated based on, the usage of the inhaler 100 (e.g. pressure metrics, airflow metrics, lung function metrics, dose confirmation information, etc.) may be computed and/or assessed via external devices as well (e.g. partially or entirely). More specifically, the wireless communication circuit 129 in the electronics module 120 may include a transmitter and/or receiver (e.g. a transceiver), as well as additional circuitry. The wireless communication circuit 129 may include, or define, the transmission module 14 of the inhaler 100.

For example, the wireless communication circuit 129 may include a Bluetooth chip set (e.g. a Bluetooth Low Energy chip set), a ZigBee chipset, a Thread chipset, etc. As such, the electronics module 120 may wirelessly provide the personalized data, such as pressure measurements, airflow metrics, lung function metrics, dose confirmation information, and/or other conditions related to usage of the inhaler 100, to an external processing module 34, such as a processing module 34 included in a smart phone 40. The personalized data may be provided in real time to the external device to enable acute risk level determination based on real-time data from the inhaler 100 that indicates time of use, how the inhaler 100 is being used, and personalized data about the subject, such as real-time data related to the subject's lung function and/or medical treatment. The external device may include software for processing the received information and for providing compliance and adherence feedback to the subject via a graphical user interface (GUI). The graphical user interface may be included in, or may define, the user interface 38 included in the system 10.

The airflow metrics may include personalized data that is collected from the inhaler 100 in real-time, such as one or more of an average flow of an inhalation/exhalation, a peak flow of an inhalation/exhalation (e.g. a maximum inhalation received), a volume of an inhalation/exhalation, a time to peak of an inhalation/exhalation, and/or the duration of an inhalation/exhalation. The airflow metrics may also be indicative of the direction of flow through the flow pathway 119. That is, a negative change in pressure may correspond to an inhalation from the mouthpiece 106, while a positive change in pressure may correspond to an exhalation into the mouthpiece 106. When calculating the airflow metrics, the electronics module 120 may be configured to eliminate or minimize any distortions caused by environmental conditions. For example, the electronics module 120 may re-zero to account for changes in atmospheric pressure before or after calculating the airflow metrics. The one or more pressure measurements and/or airflow metrics may be timestamped and stored in the memory of the electronics module 120.

In addition to the airflow metrics, the inhaler 100, or another computing device, may use the airflow metrics to generate additional personalized data. For example, the controller of the electronics module 120 of the inhaler 100 and/or the processing module 34 may translate the airflow metrics into other metrics that indicate the subject's lung function and/or lung health that are understood to medical practitioners, such as peak inspiratory flow metrics, peak expiratory flow metrics, and/or forced expiratory volume in 1 second (FEV1), for example. The processing module 34 and/or the electronics module 120 of the inhaler 100 may determine a measure of the subject's lung function and/or lung health using a mathematical model such as a regression model. The mathematical model may identify a correlation between the total volume of an inhalation and FEV1. The mathematical model may identify a correlation between peak inspiratory flow and FEV1. The mathematical model may identify a correlation between the total volume of an inhalation and peak expiratory flow. The mathematical model may identify a correlation between peak inspiratory flow and peak expiratory flow.

The battery 126 may provide power to the components of the PCB 122. The battery 126 may be any suitable source for powering the electronics module 120, such as a coin cell battery, for example. The battery 126 may be rechargeable or non-rechargeable. The battery 126 may be housed by the battery holder 124. The battery holder 124 may be secured to the PCB 122 such that the battery 126 maintains continuous contact with the PCB 122 and/or is in electrical connection with the components of the PCB 122. The battery 126 may have a particular battery capacity that may affect the life of the battery 126. As will be further discussed below, the distribution of power from the battery 126 to the one or more components of the PCB 122 may be managed to ensure the battery 126 can power the electronics module 120 over the useful life of the inhaler 100 and/or the medication contained therein.

In a connected state, the communication circuit and memory may be powered on and the electronics module 120 may be “paired” with an external device, such as a smart phone. The controller may retrieve data from the memory and wirelessly transmit the data to the external device. The controller may retrieve and transmit the data currently stored in the memory. The controller may also retrieve and transmit a portion of the data currently stored in the memory. For example, the controller may be able to determine which portions have already been transmitted to the external device and then transmit the portion(s) that have not been previously transmitted. Alternatively, the external device may request specific data from the controller, such as any data that has been collected by the electronics module 120 after a particular time or after the last transmission to the external device. The controller may retrieve the specific data, if any, from the memory and transmit the specific data to the external device.

The data stored in the memory of the electronics module 120 (e.g. the signals generated by the switch 130, the pressure measurement readings taken by the sensory system 128 and/or the airflow metrics computed by the controller of the PCB 122) may be transmitted to an external device, which may process and analyze the data to determine the usage parameters associated with the inhaler 100. Further, a mobile application residing on the mobile device may generate feedback for the user based on data received from the electronics module 120. For example, the mobile application may generate daily, weekly, or monthly report, provide confirmation of error events or notifications, provide instructive feedback to the subject, and/or the like.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

### Additional Disclosure

Also provided is a system for determining a probability that a subject having a chronic respiratory disease also has a severe acute respiratory syndrome.

An exemplary system comprises a first inhaler for delivering a rescue medicament. In this example, the first inhaler has a use determination system configured to determine a rescue inhalation performed by the subject using the first inhaler.

The system optionally also includes a second inhaler for delivering a maintenance medicament to the subject during a routine inhalation.

The exemplary system comprises a sensor system configured to measure a parameter relating to airflow during the rescue inhalation and/or during the routine inhalation using the second inhaler when such a second inhaler is included in the system.

The exemplary system further comprises a processor configured to determine a number of the rescue inhalations during a first time period, receive the parameter measured for at least some of the rescue and/or the routine inhalations, and determine the probability of the subject having the severe acute respiratory syndrome based on the number of rescue inhalations and the parameters.

Use of both the number of rescue inhalations and the parameter relating to airflow during the rescue and/or routine inhalations may thus be used to determine the probability or likelihood that the subject has the severe acute respiratory syndrome. This is because the number of uses of the rescue inhaler tends to increase with the onset of symptoms of the severe acute respiratory syndrome. Moreover, the lung function/health of the subject becomes impaired with the onset of such symptoms. This deterioration can be detected via the parameter relating to airflow measured during inhalations performed using the inhaler(s).

Thus, data from inhaler usage can be used to make at least an initial assessment of whether or not the subject is suffering from the severe acute respiratory syndrome. On the basis of this initial assessment, further diagnostic tests, such as a test configured to detect a virus in a sample from the subject, or a change in the subject's treatment, e.g. admission into a hospital or an intensive care unit (ICU), may be implemented.

In addition to the rescue medicines already described above, albuterol (also known as salbutamol), typically administered as the sulfate salt, is a preferred rescue medicine of the present disclosure.

The present disclosure is directed to a treatment approach which assesses the likelihood of the patient having contracted a severe acute respiratory syndrome, such as SARS-CoV, to allow an early intervention in the patient's treatment, thereby improving the outcome for the patient. Particular mention is made of Covid-19 as an example of a severe acute respiratory syndrome.

Provided is a system for determining a probability (or likelihood) of a severe acute respiratory syndrome in a subject. The system comprises a first inhaler for delivering a rescue medicament to the subject. The rescue medicament may be suitable for treating a worsening of respiratory symptoms, for example by effecting rapid dilation of the bronchi and bronchioles upon inhalation of the medicament. The first inhaler has a use determination system configured to determine a rescue inhalation performed by the subject using the first inhaler. The system optionally includes a second inhaler for delivering a maintenance medicament to the subject during a routine inhalation. A sensor system is configured to measure a parameter relating to airflow during the rescue inhalation and/or during the routine inhalation, when the second inhaler is included in the system.

The rescue medicament is as defined hereinabove and is typically a SABA or a rapid-onset LABA, such as formoterol (fumarate). The rescue medicine may also be in the form of a combination product, e.g. ICS-formoterol (fumarate), typically budesonide-formoterol (fumarate). Such an approach is termed “MART” (maintenance and rescue therapy). However, the presence of a rescue medicine indicates that it is a first inhaler within the meaning of the present disclosure since the presence of the rescue medicament is determinative in the probability determination. It therefore covers both a rescue medicament and a combination rescue and maintenance medicament. In contrast, the the second inhaler, when present, is only used for the maintenance aspect of the therapy and not for rescue purposes. The key difference is that the first inhaler may be used as-needed, whereas the second inhaler is intended for use at regular, pre-defined times.

The system further comprises a processor configured to determine a number of the rescue inhalations during a first time period, and receive the parameter measured for at least some of the rescue and/or routine inhalations. The processor then determines the probability of the severe acute respiratory syndrome based on the number of rescue inhalations and the parameters. Any preferred embodiments discussed in respect of the system may be applied to the methods, and vice versa.

Assessing the risk of a severe acute respiratory syndrome for a sufferer of a chronic respiratory disease, such as an asthma or COPD, can involve monitoring various subject-related and environmental factors. A challenge concerns which factors should be taken into account, and which neglected. Neglecting factors which only have a minimal or negligible influence on the risk determination may enable determination of the risk more efficiently, for example using less computational resources, such as processing resources, battery power, memory requirements, etc. Of greater importance is the requirement to improve the accuracy with which the severe acute respiratory syndrome may be determined. A more accurate risk determination may facilitate a more effective warning system so that the appropriate clinical intervention may be delivered to the subject. Thus, more accurate assessment of the probability that the subject has a severe acute respiratory syndrome may have the potential to guide intervention for a subject at acute risk.

For a higher probability of the subject having a severe acute respiratory syndrome, a step change in the treatment regimen may, for instance, be justified to a regimen configured for subjects at greater acute risk. For example, the subject may be admitted into an intensive care unit (ICU), and/or provided with oxygen or ventilator therapy. Alternatively, in the case of a lower probability of a severe acute respiratory syndrome, enhanced accuracy of the probability determination may be used as guidance to justify downgrading or even removal of an existing treatment regimen, such as discharge of the subject from the ICU or the hospital.

The probability determination is based both on the number of rescue inhalations of a rescue medicament performed by the subject during a (first) time period and a parameter relating to airflow during inhalations of a rescue and/or maintenance medicament.

The first time period corresponds to the sample period over which the number of rescue inhalations is counted. The first time period may be, for example, 1 to 15 days. This sample period may be selected such that the period allows for an indicative number of rescue inhalations to occur. A sample period which is too short may not permit sufficient inhalation data to be collected for reliable prediction, whilst a sample period which is too long may have an averaging effect which renders shorter term trends which are of diagnostic or predictive significance less distinguishable.

Use of both the number of rescue inhalations and the parameter may lead to a more accurate predictive model than, for example, a model which neglects either one of these factors.

Basing the determination on the number of rescue inhalations may mean that the model uses the absolute number of rescue inhalations during the first time period and/or one or more trends based on the number of rescue inhalations. Such trends are not the number of rescue inhalations per se, but are variations in the number of rescue inhalations.

The trends based on the number of rescue inhalations may, for example, include the number of inhalations performed during a particular period in the day. The number of night-time inhalations may therefore, for instance, be included as a factor in the number of inhalations. The processor may, for example, be equipped with suitable clock functionality in order to record such time of day rescue medicament use.

The probability determination is also based on the parameter relating to airflow during the rescue inhalation and/or during the routine inhalation using the second inhaler when present. The parameter may correspond to a single factor relating to airflow during inhalation or may include a plurality of such factors. For example, the parameter may be at least one of a peak inhalation flow, an inhalation volume, an inhalation duration, and an inhalation speed. The time to peak inhalation flow may, for example, provide a measure of the inhalation speed.

Basing the determination on the parameters may mean that the model uses the one or more factors relating to airflow during the inhalations and/or one or more trends associated with the respective factor or factors. Such trends correspond to variations in the respective factor(s).

When the chronic respiratory disease is asthma, the number of rescue inhalations, including trends relating to rescue inhaler usage, may be more significant in the probability determination than the parameter relating to airflow during the inhalations. The parameter may still be a significant factor in determining the probability, but may exert less overall influence on the probability than the number of rescue inhalations. Accordingly, further enhancement of the accuracy of the probability determination, at least in the case of asthma, stems from weighting the predictive model such that the number of rescue inhalations is more significant in the probability determination than the parameter.

When the chronic respiratory disease is asthma, the predictive model may have, for example, a first weighting coefficient associated with the number of rescue inhalations and a second weighting coefficient associated with the parameters. When standardized to account for the different units used to quantify the number of rescue inhalations (or related trends of rescue medicament use) and the parameters, the first weighting coefficient may be larger than the second weighting coefficient, thereby ensuring that the number of rescue inhalations is more significant in the probability determination than the parameter when the chronic respiratory disease is asthma.

Thus, in the case of the chronic respiratory disease being asthma, the first weighting coefficient may weight the absolute number of rescue inhalations and/or the one or more trends based on the number of rescue inhalations.

For example, the number of rescue inhalations (e.g. including any related trends) may have a significance/importance (e.g. weight) in the model (relative to the other factors) of 40% to 95%, preferably 55% to 95%, more preferably 60% to 85%, and most preferably 60% to 80%, e.g. about 60% or about 80%.

In the case of the chronic respiratory disease being asthma, the second weighting coefficient may weight the one or more factors relating to airflow during the inhalations and/or the one or more trends associated with the respective factor or factors.

For example, the inhalation parameters (e.g. including any related trends) may have a significance/importance (e.g. weight) in the model of 2% to 49% or 2% to 30%, preferably 2% to 45%, more preferably 5% to 40%, and most preferably 10% to 35%, e.g. about 10% or about 35%.

In some embodiments, a biometric parameter may be included in the weighted model to further improve its accuracy. In such embodiments, the processor may, for example, be configured to receive the biometric parameter. A data input unit may, for instance, be included in the system to enable the subject and/or healthcare provider to input the biometric parameter.

For the predictive model when the chronic respiratory disease is asthma, the model may, for example, be weighted such that the biometric parameter has a lower significance than the number of rescue inhalations in the probability determination. In other words, a third weighting coefficient may be associated with the biometric parameter (or biometric parameters), which third weighting coefficient may be smaller than the first weighting coefficient associated with the number of rescue inhalations. The third weighting coefficient may be larger or smaller than the second weighting coefficient associated with the parameter relating to airflow.

Preferably, when the chronic respiratory disease is asthma the third weighting coefficient is smaller than the second weighting coefficient. In order of predictive power, the rescue medicament use may thus have the greatest influence, then the inhalation parameter, and then the biometric parameter.

The biometric parameter may be, for instance, one or more selected from body weight, height, body mass index, blood pressure, including systolic and/or diastolic blood pressure, sex, race, age, smoking history, sleep/activity patterns, exacerbation history, other treatments or medicaments administered to the subject, etc. In an embodiment, the biometric parameter includes age, body mass index and exacerbation history. In a preferred embodiment, the biometric parameter includes exacerbations and medical history, body mass index, and blood pressure, for example systolic and/or diastolic blood pressure.

For the predictive model when the chronic respiratory disease is asthma, the biometric parameter may have a significance/importance (e.g. weight) in the model of 1% to 15%, preferably 1% to 12%, more preferably 3% to 10%, and most preferably 4% to 10%, e.g. about 5% or about 8%.

In a non-limiting example where the chronic respiratory disease is asthma, the number of rescue inhalations (e.g. including any related trends) has a significance/importance (e.g. weight) in the model (relative to the other factors) of 40% to 95%, preferably 55% to 90%, more preferably 60% to 85%, and most preferably 60% to 80%, e.g. about 60% or about 80%; the inhalation parameters (e.g. including any related trends) has a significance/importance (e.g. weight) in the model of 2% to 49%, preferably 2% to 45%, more preferably 5% to 40%, and most preferably 10% to 35%, e.g. about 10% or about 35%; and the biometric parameter has a significance/importance (e.g. weight) in the model of 1% to 15%, preferably 1% to 12%, more preferably 3% to 10%, and most preferably 4% to 10%, e.g. about 5% or about 8%.

More generally for the predictive model when the chronic respiratory disease is asthma, the number of rescue inhalations (e.g. including any related trends in the number of rescue inhalations) may be the most significant factor in the probability determination.

When the chronic respiratory disease is COPD, the predictive model may have, for example, a first weighting coefficient associated with the parameter, and a second weighting coefficient associated with the number of rescue inhalations. When standardized to account for the different units used to quantify the number of rescue inhalations (or related trends of rescue medicament use) and the parameters, the first weighting coefficient may be larger than the second weighting coefficient, thereby ensuring that the parameter is more significant in the probability determination than the number of rescue inhalations.

The parameter may correspond to a single factor relating to airflow during inhalation or may include a plurality of such factors. For example, the parameter may be at least one of a peak inhalation flow, an inhalation volume, an inhalation duration, and an inhalation speed. The time to peak inhalation flow may, for example, provide a measure of the inhalation speed, as previously described.

The first weighting coefficient in the model for COPD may weight the one or more factors relating to airflow during the inhalations and/or the one or more trends associated with the respective factor or factors.

When the chronic respiratory disease is COPD, the parameter relating to airflow during the rescue inhalations and/or during the routine inhalations (e.g. including any related trends) may have a significance/importance (e.g. weight) in the model (relative to the other factors) of 55% to 95%, preferably 65% to 90%, and most preferably 75% to 85%, e.g. about 80%.

The second weighting coefficient in the model for COPD may weight the absolute number of rescue inhalations and/or the one or more trends based on the number of rescue inhalations.

When the chronic respiratory disease is COPD, the number of rescue inhalations (e.g. including any related trends) may have a significance/importance (e.g. weight) in the model of 2% to 30%, preferably 5% to 25%, and most preferably 10% to 20%, e.g. about 15%.

In some embodiments, a biometric parameter may be included in the weighted model for COPD to further improve its accuracy. In such embodiments, the processor may, for example, be configured to receive the biometric parameter. A data input unit may, for instance, be included in the system to enable the subject and/or healthcare provider to input the biometric parameter.

When the chronic respiratory disease is COPD, the model may, for example, be weighted such that the biometric parameter has a lower significance than the parameter relating to airflow during inhalations in the probability determination. In other words, a third weighting coefficient may be associated with the biometric parameter (or biometric parameters), which third weighting coefficient may be smaller than the first weighting coefficient associated with the parameter. The third weighting coefficient may be larger or smaller than the second weighting coefficient associated with the number of rescue inhalations.

When the chronic respiratory disease is COPD, the third weighting coefficient is preferably smaller than the second weighting coefficient. In order of predictive power, the parameter relating to airflow during inhalations may thus have the greatest influence, then the number of rescue inhalations, and then the biometric parameter.

When the chronic respiratory disease is COPD the biometric parameter may have a significance/importance (e.g. weight) in the model of 1% to 12%, preferably 3% to 10%, and most preferably 4% to 6%, e.g. about 5%.

More generally for the predictive model when the chronic respiratory disease is COPD, the parameter relating to airflow (e.g. including any related trends in the parameter) may be the most significant factor in the probability determination.

Irrespective of the respiratory disease, additional data sources may also be added to the model, such as environmental data relating to the weather or pollution levels. Such additional data may be weighted such as to have less significance on the probability determination than the number of rescue inhalations and the inhalation parameter data.

The model may be a linear model or may be a non-linear model. The model may be, for instance, a machine learning model. A supervised model, such as a supervised machine learning model, may, for example, be used. Irrespective of the specific type of model employed, the model is constructed to be more sensitive, i.e. responsive, to the number of rescue inhalations than the inhalation parameters for asthma, and more sensitive to the inhalation parameters than the number of rescue inhalations for COPD, as previously described. It is this sensitivity which may correspond to the “weighting” of the weighted model.

In a non-limiting example, the model is constructed using a decision trees technique. Other suitable techniques, such as building a neural network or a deep learning model may also be contemplated by the skilled person.

Irrespective of the chronic respiratory disease, the processor of the system may, in a non-limiting example, determine the probability based on the number of inhalations, the inhalation parameters and an indication of a status of the respiratory disease being experienced by the subject. The inclusion of the indication in the prediction may enhance the accuracy of the prediction. This is because the user-inputted indication may assist to validate or enhance the predictive value of the probability assessment relative to that derived from, for example, consideration of the number of inhalations and the inhalation parameters without such a user-inputted indication.

In an embodiment, the processor determines an initial probability of the subject having a severe acute respiratory syndrome based on the recorded inhalation or inhalations, and the received inhalation parameter or parameters, but not on the indication. The initial probability may, for example, be calculated using a weighted model, e.g. as described above. The probability, i.e. the overall probability, may then be determined based on the inhalation(s), the parameter(s) and the received indication of the status of the respiratory disease being experienced by the subject. For example, the overall probability may be determined based on the initial probability and the received indication.

By including the user-inputted indication in the probability determination, one or more of: positive and negative predictive values, the sensitivity of the prediction, i.e. the capability of the system/method to correctly identify those at risk (true positive rate), and the specificity of the prediction, i.e. the capability of the system/method to correctly identify those not at risk (true negative rate), may be enhanced.

The processor may, for example, be configured to control a user interface to issue a prompt to the user so that the user inputs the indication. The prompt may be issued based on the initial probability determined from the inhalation(s) and the inhalation parameter(s), but not on the indication. For example, the prompt may be issued based on the initial probability reaching or exceeding a predetermined threshold. In this manner, the user may be prompted by the system to input the indication on the basis of the initial probability signaling a potential severe acute respiratory syndrome. By the user then inputting the indication, the (overall) probability which also takes account of the indication may assist to confirm or validate the initial probability.

This may be, for instance, regarded as an “analytics data driven” use of the indication: the user input is requested when the inhalation and inhalation parameter data indicate possible worsening of the subject's condition.

The user interface may, for example, prompt the user or subject to provide the indication via a pop-up notification link to complete a short questionnaire. The logic determining when this pop-up notification is provided may, for example, be driven by shifts in key variables, such as changes in the number and/or time of rescue and/or controller inhalations, and inhalation parameters.

Alternatively or additionally, the system may be configured to receive the indication when the user opts to input the indication via the user interface. For example, when the healthcare provider decides that the indication may usefully enhance the initial probability determination, the subject may be requested to input the indication. This may, for instance, be regarded as an “on request” use of the indication: the request being made by the patient or his/her physician, e.g. prior to or during an assessment by the healthcare professional.

In this manner, the user may only be prompted to input the indication when this is deemed necessary by the system and/or healthcare provider. This may advantageously reduce the burden on the subject, and render it more likely that the subject will input the indication when asked or prompted to do so, i.e. when such input would be desirable in relation to monitoring the subject's respiratory condition. Inputting the indication in these embodiments may thus be more likely than the scenario in which the subject is routinely prompted to input the indication.

In an embodiment, the user interface is configured to provide a plurality of user-selectable respiratory disease status options. In this case, the indication is defined by user-selection of at least one of the status options.

For example, the user interface may display a questionnaire comprising questions whose answers correspond to the indication. The user, e.g. the subject or his/her health care provider, may input the answers to the questions using the user interface.

The questionnaire may be relatively short, i.e. with relatively few questions, in order to minimize burden on the subject. The number and nature of the questions may nevertheless be such as to ensure that the indication enables the probability determination to be enhanced relative to the scenario where no indication is inputted.

More generally, the object of the questionnaire is to ascertain a contemporaneous or relatively recent (e.g. within the past 24 hours) indication in order to obtain “in the moment” understanding of the subject's well-being (in respect of their respiratory disease) with a few timely questions which are relatively quickly answered. The questionnaire may be translated into the local language of the subject.

Conventional control questionnaires, and especially the most established being ACQ/T (Asthma Control Questionnaire/Test) in asthma, or CAT (COPD Assessment Test) in COPD tend to focus on patient recall of symptoms in the past. Recall bias, and a focus on the past instead of the present is likely to negatively influence their value for the purposes of predictive analysis.

The following is provided by way of non-limiting example of such a questionnaire. The subject may select from the following status options for each question: All of the time (5); Most of the time (4); Some of the time (3); A little (2); None (1).
1. How ‘often are you experiencing’, or ‘Rate your’ shortness of breath?
2. How ‘often are you experiencing’, or ‘Rate your’ coughing?
3. How ‘often are you experiencing’, or ‘Rate your’ wheezing?
4. How ‘often are you experiencing’, or ‘Rate your’ chest tightness?
5. How ‘often are you experiencing’, or ‘Rate your’ night symptoms/affecting sleep?
6. How ‘often are you experiencing’, or ‘Rate your’ limitation at work, school or home?

An alternative example questionnaire is also provided:
1. Are you having more respiratory symptoms than usual (Y/N)? If yes:
2. More chest tightness or shortness of breath (Y/N)?
3. More cough (Y/N)?
4. More wheezing (Y/N)?
5. Is it affecting your sleep (Y/N)?
6. Is it limiting your activities at home/work/school (Y/N)?

The answers to the questions may, for example, be used to calculate a score, which score is included in, or corresponds to, the indication of the status of the respiratory disease being experienced by the subject.

In an embodiment, the user interface is configured to provide the status options in the form of selectable icons, e.g. emoji-type icons, checkboxes, a slider, and/or a dial. In this way, the user interface may provide a straightforward and intuitive way of inputting the indication of the status of the respiratory disease being experienced by the subject. Such intuitive inputting may be particularly advantageous when the subject himself/herself is inputting the indication, since the relatively facile user-input may be minimally hampered by any worsening of the subject's condition.

Any suitable user interface may be employed for the purpose of enabling user-input of the indication of the status of the respiratory disease being experienced by the subject. For example, the user interface may comprise or consist of a (first) user interface of a user device. The user device may be, for example, a personal computer, a tablet computer, and/or a smart phone. When the user device is a smart phone, the (first) user interface may, for instance, correspond to the touchscreen of the smart phone.

In an embodiment, the processor of the system may be at least partly included a (first) processor included in the user device. Alternatively or additionally, the first inhaler and/or the second inhaler may, for example, include a (second) processor, and the processor of the system may be at least partly included in the (second) processor included in the inhaler.

A method is provided for determining a probability that a subject having a chronic respiratory disease also has a severe acute respiratory syndrome, the method comprising: receiving a number of rescue inhalations of a rescue medicament prescribed for the chronic respiratory disease, the rescue inhalations being performed by the subject during a first time period; receiving a parameter relating to airflow during at least some of the rescue inhalations and/or during routine inhalations performed by the subject of a maintenance medicament; and determining the probability of the subject having the severe acute respiratory syndrome based on the number of rescue inhalations and the parameters.

Further provided is a method for treating a severe acute respiratory syndrome in a subject suffering from a chronic respiratory disease, the method comprising: performing the above-described method; determining whether the probability reaches or exceeds a predetermined upper threshold; or determining whether the probability reaches or is lower than a predetermined lower threshold; and treating the severe acute respiratory syndrome based on the probability reaching or exceeding the predetermined upper threshold; or based on the probability reaching or being lower than the predetermined lower threshold.

The treatment may comprise modifying an existing treatment. The existing treatment may comprise a first treatment regimen, and the modifying the existing treatment of the asthma may comprise changing from the first treatment regimen to a second treatment regimen based on the probability reaching or exceeding the predetermined upper threshold, wherein the second treatment regimen is configured for higher risk of the severe acute respiratory syndrome than the first treatment regimen.

In an embodiment, the second treatment regimen comprises admitting the subject into an intensive care unit.

The more accurate probability determination using the weighted model may facilitate a more effective warning system so that the appropriate clinical intervention may be delivered to the subject. Thus, more accurate assessment of the probability may have the potential to guide intervention for a subject at acute risk.

As the serious acute respiratory syndrome progresses, interstitial changes in the lungs may spread and, particularly once the subject is admitted into the ICU, there may be a greater risk of the subject developing acute respiratory distress syndrome (ARDS). ARDS may ultimately affect the alveoli, and the natural surfactant in the lungs.

Thus, in a non-limiting example, the second treatment regime comprises administering an exogenous surfactant and/or a cholesterol sequestering agent, such as a cyclodextrin, to the subject. The method of administration of such an exogenous surfactant or cholesterol sequestering agent is not particularly limited. This may, for instance, be administered in nebulized form via a ventilator system when the subject is required to be intubated for ventilation therapy.

In an embodiment, the second treatment regimen comprises administering a (further) medication to the subject, such as dexamethasone, or a biologics medication.

In the case of dexamethasone, administration of this medicament may only be appropriate when the subject's symptoms are relatively serious. Dexamethasone can be harmful to subjects whose symptoms are milder. The probability determination of the present disclosure may provide a metric which justifies administration of dexamethasone to the subject, i.e. when the predetermined upper threshold is reached or exceeded.

The relatively high cost of biologics medications means that stepping up the subject's treatment to include administering of a biologics medication tends to require careful consideration and justification. The systems and methods according to the present disclosure may provide a reliable metric to justify administering of a biologics medication. For example, should the determined probability reach or surpass an upper threshold indicative of a high probability of the severe acute respiratory syndrome, the administering of the biologics medication may be quantitatively justified, and the biologics medication may be administered accordingly.

More generally, the biologics medication may comprise one or more of omalizumab, mepolizumab, reslizumab, benralizumab and dupilumab.

In another embodiment, the treating comprises switching the subject from a first treatment regimen to a third treatment regimen based on the probability reaching or being lower than the predetermined lower threshold, wherein the third treatment regimen is configured for lower risk of the severe acute respiratory syndrome than the first treatment regimen.

In a non-limiting example, the first treatment regimen comprises treating the subject in an intensive care unit or hospital, and the third treatment regimen comprises treating the subject in a setting which is outside the intensive care unit or hospital respectively. Alternatively or additionally, administration of a medicament prescribed to treat the severe acute respiratory syndrome, such as dexamethasone, may be ceased, or a lower dose may be prescribed, on the basis of the probability reaching or being lower than the predetermined lower threshold.

A method is provided for diagnosing a severe acute respiratory syndrome the method comprising: performing the above-described method for determining the probability that a subject having a chronic respiratory disease also has a severe acute respiratory syndrome; determining whether the probability reaches or exceeds a predetermined upper threshold indicative of the severe acute respiratory syndrome; and diagnosing the severe acute respiratory syndrome based on the probability reaching or exceeding the predetermined upper threshold.

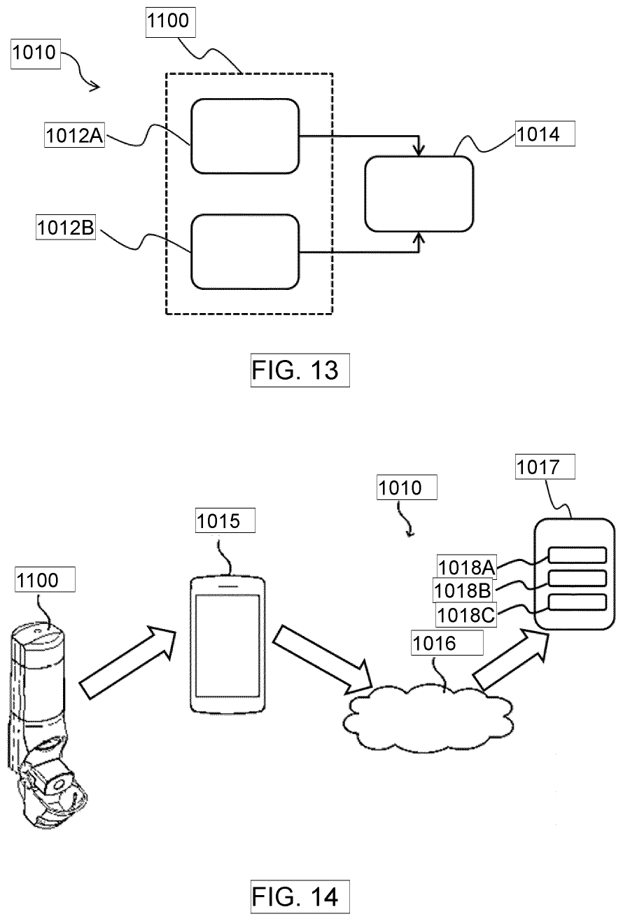

FIG. 13 shows a block diagram of a system 1010 according to an embodiment. The system 1010 comprises a first inhaler 1100 and a processor 1014. The first inhaler 1100 may be used to deliver a rescue medicament, such as a SABA, to the subject. The SABA may include, for example, albuterol. The first inhaler 1100 may include a sensor system 1012A and/or a use determination system 1012B.

The system 1010 may, for example, be alternatively termed “an inhaler assembly”.

The first inhaler may, for example, be alternatively termed “a rescue inhaler”.

The second inhaler may, for example, be alternatively termed “a maintenance inhaler” or “a controller inhaler”.

The number of rescue inhalations is determined by a use determination system 1012B included in the first inhaler 1100.

A sensor system 1012A may be configured to measure the parameter. The sensor system 1012A may, for example, comprise one or more sensors, such as one or more pressure sensors, temperature sensors, humidity sensors, orientation sensors, acoustic sensors, and/or optical sensors. The pressure sensor(s) may include a barometric pressure sensor (e.g. an atmospheric pressure sensor), a differential pressure sensor, an absolute pressure sensor, and/or the like. The sensors may employ microelectromechanical systems (MEMS) and/or nanoelectromechanical systems (NEMS) technology.

A pressure sensor(s) may be particularly suitable for measuring the parameter, since the airflow during inhalation by the subject may be monitored by measuring the associated pressure changes. As is explained in greater detail above with reference to FIGS. 8-12, a pressure sensor may be, for instance, located within or placed in fluid communication with a flow pathway through which air and the medicament is drawn by the subject during inhalation. Alternative ways of measuring the parameter, such as via a suitable flow sensor, will also be apparent to the skilled person.

Alternatively or additionally, the sensor system 1012A may comprise a differential pressure sensor. The differential pressure sensor may, for instance, comprise a dual port type sensor for measuring a pressure difference across a section of the air passage through which the subject inhales. A single port gauge type sensor may alternatively be used. The latter operates by measuring the difference in pressure in the air passage during inhalation and when there is no flow. The difference in the readings corresponds to the pressure drop associated with inhalation.

Whilst not shown in FIG. 13, the system 1010 may further comprise a second inhaler for delivering a maintenance medicament to the subject during a routine inhalation. The second inhaler may include a sensor system 1012A and/or a use determination system 1012B that is distinct from the sensor system 1012A and/or the use determination system 1012B of the first inhaler 1100. The sensor system 1012A of the second inhaler may be configured to measure the parameter during the routine inhalation. For example, the sensor system 1012A may include a further pressure sensor, such as a further microelectromechanical system pressure sensor or a further nanoelectromechanical system pressure sensor, in order to measure the parameter during maintenance medicament inhalation.

In this manner, inhalation of either or both the rescue and the maintenance medicaments may be used to gather information relating to the subject's lung function and/or lung health. When both the first and second inhalers are used, the accuracy with which the probability of the subject having the severe acute respiratory syndrome may be improved by the additional inhalation data supplied by monitoring both routine and rescue medicament inhalations.

Each inhalation may be associated with a decrease in the pressure in the airflow channel relative to when no inhalation is taking place. The point at which the pressure is at its lowest may correspond to the peak inhalation flow. The sensor system 1012A may detect this point in the inhalation. The peak inhalation flow may vary from inhalation to inhalation, and may depend on the clinical condition of the subject. Lower peak inhalation flows may, for example, be recorded when the subject has a severe acute viral respiratory syndrome. The term “minimum peak inhalation flow” as used herein may mean the lowest peak inhalation flow recorded for inhalations performed using the first and/or second inhaler during a (second) time period.

The pressure change associated with each inhalation may alternatively or additionally be used to determine an inhalation volume. This may be achieved by, for example, using the pressure change during the inhalation measured by the sensor system 1012A to first determine the flow rate over the time of the inhalation, from which the total inhaled volume may be derived. Lower inhalation volumes may be recorded when, for instance, the subject has a severe acute respiratory syndrome, since the subject's capacity to inhale may be diminished. The term “minimum inhalation volume” as used herein may mean the lowest inhalation volume recorded for inhalations performed using the first and/or second inhaler during a (third) time period.

The pressure change associated with each inhalation may alternatively or additionally be used to determine an inhalation duration. The time may be recorded, for example, from the first decrease in pressure measured by the pressure sensor 1012A, coinciding with the start of the inhalation, to the pressure returning to a pressure corresponding to no inhalation taking place. Lower inhalation durations may be recorded when, for instance, the subject has a severe acute respiratory syndrome, since the subject's capacity for inhaling for longer may be diminished. The term “minimum inhalation duration” as used herein may mean the shortest inhalation duration recorded for inhalations performed using the first and/or second inhaler during a (fourth) time period.

In an embodiment, the parameter includes the time to peak inhalation flow, e.g. as an alternative or in addition to the peak inhalation flow, the inhalation volume and/or the inhalation duration. This time to peak inhalation flow parameter may be recorded, for example, from the first decrease in pressure measured by the sensor system 1012A, coinciding with the start of the inhalation, to the pressure reaching a minimum value corresponding to peak flow. A subject having a severe acute respiratory syndrome may take a longer time to achieve peak inhalation flow.

In a non-limiting example, the first and/or second inhalers may be configured such that, for a normal inhalation, the respective medicament is dispensed during approximately 0.5 s following the start of the inhalation. A subject's inhalation only reaching peak inhalation flow after the 0.5 s has elapsed, such as after approximately 1.5 s, may be partially indicative of the subject having a severe acute respiratory syndrome.

The use determination system 1012B is configured to register inhalation(s) by the subject (e.g. each rescue inhalation by the subject when the inhaler is a rescue inhaler, or each maintenance inhalation by the subject when the inhaler is a maintenance inhaler). In a non-limiting example, the first inhaler 1100 may comprise a medicament reservoir (not shown in FIG. 13), and a dose metering assembly (not shown in FIG. 13) configured to meter a dose of the rescue medicament from the reservoir. The use determination system 1012B may be configured to register the metering of the dose by the dose metering assembly, each metering being thereby indicative of the rescue inhalation performed by the subject using the first inhaler 1100. Accordingly, the inhaler 1100 may be configured to monitor the number of rescue inhalations of the medicament, since the dose must be metered via the dose metering assembly before being inhaled by the subject. One non-limiting example of the metering arrangement is explained in greater detail above with reference to FIGS. 8-12.

Alternatively or additionally, the use determination system 1012B may register each inhalation in different manners and/or based on additional or alternative feedback that are apparent to the skilled person. For example, the use determination system 1012B may be configured to register an inhalation by the subject when the feedback from the sensor system 1012A indicates that an inhalation by the user has occurred (e.g. when a pressure measurement or flow rate exceeds a predefined threshold associated with a successful inhalation). Further, in some examples, the use determination system 1012B may be configured to register an inhalation when a switch of the inhaler or a user input of an external device (e.g. touchscreen of a smartphone) is manually actuated by the subject prior to, during or after inhalation.

A sensor (e.g. a pressure sensor) may, for example, be included in the use determination system 1012B in order to register each inhalation. In such an example, the use determination system 1012B and the sensor system 1012A may employ respective sensors (e.g. pressure sensors), or a common sensor (e.g. a common pressure sensor) which is configured to fulfil both use-detecting and inhalation parameter sensing functions.

When a sensor is included in the use determination system 1012B, the sensor may, for instance, be used to confirm that, or assess the degree to which, a dose metered via the dose metering assembly is inhaled by the user, as is described above in greater detail with reference to FIGS. 8-12.

In an embodiment, the sensor system 1012A and/or the use determination system 1012B includes an acoustic sensor, as described above with regard to use determination system 12. The respective inhaler may comprise a capsule, as described above in connection with the acoustic sensor of use determination system 12, for deriving use and/or inhalation parameter data.

An algorithm may, for example, be used to interpret the acoustic data in order to determine use data (when the acoustic sensor is included in the use determination system 1012B) and/or the parameter relating to airflow during the inhalation (when the acoustic sensor is included in the sensor system 1012A).

For instance, an algorithm as described by Colthorpe et al. in “Adding Electronics to the Breezhaler: Satisfying the Needs of Patients” (Respiratory Drug Delivery 2018; page 71-79) may be used. Once the generated sound is detected, the algorithm may process the raw acoustic data to generate the use and/or inhalation parameter data.

The processor 1014 included in the system 1010 determines the number of rescue and/or routine inhalations during the first time period and receives the parameter measured for each of the rescue and/or routine inhalations. As schematically shown in FIG. 13 by the arrows between the sensor system 1012A and the processor 1014, and between the use determination system 1012B and the processor 1014, the processor 1014 may receive the inhalation and parameter data from the use determination system 1012B and the sensor system 1012A respectively. The processor 1014 is further configured to determine the probability that the subject has a severe acute respiratory disease based on the number of rescue inhalations and the parameters, as previously described.

In a non-limiting example, the processor 1014 may be provided separately from the respective first and/or second inhaler(s), in which case the processor 1014 receives the number of rescue inhalations and parameter data transmitted to it from the sensor system 1012A and the use determination system 1012B of the first and/or second inhalers. By processing the data in such an external processing unit, such as in the processing unit of an external device, or in a server, e.g. a remote server, the battery life of the inhaler may be advantageously preserved.

In an alternative non-limiting example, the processor 1014 may be an integral part of the first and/or second inhaler, for example contained within a main housing or top cap (not shown in FIG. 13) of the first and/or second inhaler. In such an example, connectivity to an external device need not be relied upon, since the probability determination may be performed exclusively within the first and/or second inhaler. The first and/or second inhaler may, for instance, include a suitable user interface, such as a light or lights, screen, loudspeaker, etc., for communicating the result of the probability determination to the subject. Rather than communicating the probability as a number, more intuitive means of communicating the risk to the subject may in some examples be used, such as using a light of different colors depending on the determined probability.

It may also be contemplated that some of the functions of the processor 1014 may be performed by an internal processing unit included in the first and/or second inhaler and other functions of the processor, such as the probability determination itself, may be performed by the external processing unit.

More generally, the system 1010 may include, for example, a communication module (not shown in FIG. 13) configured to communicate the determined probability to the subject and/or a healthcare provider, such as a clinician. The subject and/or the clinician may then take appropriate steps based on the determined probability. When, for instance, a smart phone processing unit is included in the processor, the communication functions of the smart phone, such as SMS, email, Bluetooth®, etc., may be employed to communicate the determined probability to the healthcare provider.

FIG. 14 shows a non-limiting example of a system 1010 for determining a probability that a subject has a severe acute respiratory syndrome. The weighted model, which may be alternatively termed a severe acute respiratory syndrome prediction model, may be used to determine the probability and the result may then be provided to the subject, caregiver and/or healthcare provider.

The example system 1010 includes the first inhaler 1100, an external device 1015 (e.g. a mobile device), a public and/or private network 1016 (e.g. the Internet, a cloud network, etc.), and a personal data storage device 1017. The external device 1015 may, for example, include a smart phone, a personal computer, a laptop, a wireless-capable media device, a media streaming device, a tablet device, a wearable device, a Wi-Fi or wireless-communication-capable television, or any other suitable Internet Protocol-enabled device. For example, the external device 1015 may be configured to transmit and/or receive RF signals via a Wi-Fi communication link, a Wi-MAX communications link, a Bluetooth® or Bluetooth® Smart communications link, a near field communication (NFC) link, a cellular communications link, a television white space (TVWS) communication link, or any combination thereof. The external device 1015 may transfer data through the public and/or private network 1016 to the personal data storage device 1017.

The first inhaler 1100 may include a communication circuit, such as a Bluetooth® radio, for transferring data to the external device 1015. The data may include the abovementioned inhalation and parameter data.

The first inhaler 1100 may also, for example, receive data from the external device 1015, such as, for example, program instructions, operating system changes, dosage information, alerts or notifications, acknowledgments, etc.

The external device 1015 may include at least part of the processor 1014, and thereby process and analyze the inhalation and parameter data. For example, the external device 1015 may process the data such as to determine the probability that a subject has a severe acute respiratory syndrome, as represented by block 1018A, and provide such information to the personal data storage device 1017 for remote storage thereon.

In some non-limiting examples, the external device 1015 may also process the data to identify no inhalation events, low inhalations events, good inhalation events, excessive inhalation events and/or exhalation events, as represented by block 1018B. The external device 1015 may also process the data to identify underuse events, overuse events and optimal use events, as represented by block 1018C. The external device 1015 may, for instance, process the data to estimate the number of doses delivered and/or remaining and to identify error conditions, such as those associated with a timestamp error flag indicative of failure of the subject to inhale a dose of the medicament which has been metered by the dose metering assembly. The external device 1015 may include a display and software for visually presenting the usage parameters through a GUI on the display. The usage parameters may be stored as personalized data that may be stored for later determining the probability that a subject has a severe acute respiratory syndrome based on real-time data.

Although illustrated as being stored on the personal data storage device 1017, in some examples, at least some of the determined probability, as represented by block 1018A, the no inhalation events, low inhalations events, good inhalation events, excessive inhalation events and/or exhalation events, as represented by block 1018B, and/or the underuse events, overuse events and optimal use events, as represented by block 1018C, may be stored on the external device 1015.

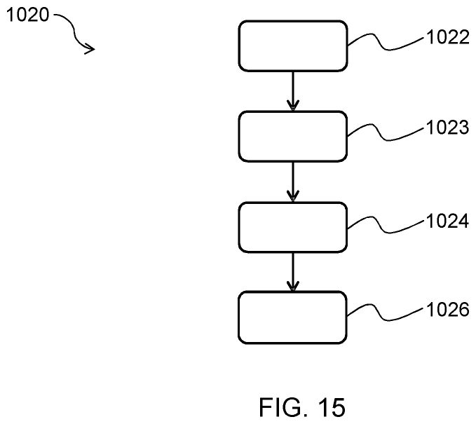

FIG. 15 shows a flowchart of a method 1020 according to an embodiment. The method 1020 may be performed by a system, such as the system 1010 illustrated in FIGS. 13 and/or 14. For example, one or more of the first and/or second inhaler, the external device 1015, and/or the personal data storage device 1017 may be configured to perform the entirety of or a portion of the method 1020. That is, any combination of the steps 1022, 1024, and 1026 may be performed by any combination of the first inhaler, the second inhaler, the external device 1015, and/or the personal data storage device 1017. Further, it should be appreciated that the steps 1022 and 1024 may be performed in any chronological order.

The method 1020 comprises determining 1022 a number of rescue inhalations of a rescue medicament performed by a subject during a first time period. In step 1024 a parameter relating to airflow during at least some, e.g. each, of the rescue and/or routine inhalations is measured. In step 1026, the probability of the subject having the severe acute respiratory syndrome is determined based on said number of rescue inhalations and said parameters.

Although not illustrated by in the method 1020, the system 1010 may be configured to notify the user if the probability exceeds or is lower than a threshold. For example, the system 1010 may be configured to determine whether the probability reaches or exceeds a predetermined upper threshold and/or reaches or is lower than a predetermined lower threshold. In response, the system 1010 may be configured to treat the patient, for example, by initiating a switch (e.g. through a message to the patient's healthcare provider) of the patient's treatment regimen to a treatment regimen that is configured for higher (or lower) probability of the severe acute respiratory syndrome than the original treatment regimen.

The system 1010 may notify the user of their probability through one or more techniques. For example, the system 1010 may be configured to display a message on the display of the external device 1015, send a message to a health-care provider or third party associated with the user, cause an indicator (e.g. light or speaker) of the inhaler 1100 to notify the user, etc.

In the non-limiting example shown in FIG. 15, the method further comprises receiving 1023 an input of an indication of a status of the respiratory disease being experienced by the subject. This input may then be used to enhance the determination of the probability that the subject is also suffering from a severe acute respiratory syndrome, as previously described.

In an embodiment, the method 1020 comprises issuing a prompt to the user so that the user inputs the indication. The prompt may be issued based on the initial probability determined from the inhalation(s) and the inhalation parameter(s), but not on the indication. For example, the prompt may be issued based on the initial probability reaching or exceeding a predetermined threshold. In this manner, the user may be prompted by the system to input the indication on the basis of the initial probability signaling a potential severe acute respiratory syndrome. By the user then inputting the indication, the (overall) probability which also takes account of the indication may assist to confirm or validate the initial probability.

More generally, the method 1020 may further comprise providing a first inhaler for delivering the rescue medicament to the subject, the first inhaler having a use determination system configured to determine the inhalation performed by the subject using the first inhaler.

The number of rescue inhalations may be determined and/or the parameter may be measured by the use determination system and/or the sensor system respectively included in the first inhaler for delivering the rescue medication. The sensor system may alternatively or additionally measure the parameter related to airflow during a routine inhalation of a maintenance medicament using a second inhaler, as previously described.

More generally, the first time period over which the number of rescue inhalations is to be determined may be 1 to 15 days, such as 3 to 8 days. Monitoring the number of rescue inhalations over such a first time period may be particularly effective in the determination of the probability of the subject having the severe acute respiratory syndrome.

When the parameter includes the peak inhalation flow, the method 1020 may further comprise determining a peak inhalation flow, such as a minimum or average peak inhalation flow from peak inhalation flows measured for inhalations performed during a second time period. The term “second” in relation to the second time period is to distinguish the period for sampling the peak inhalation flows from the first time period during which the number of rescue inhalations are sampled. The second time period may at least partially overlap with the first time period, or the first and second time periods may be concurrent.

The step 1026 of determining the probability may thus be partially based on the minimum or average peak inhalation flow. The second time period may be, for instance, 1 to 5 days, such as 1 day. The second time period may be selected according to the time required to gather peak inhalation flow data of suitable indicative value, in a manner analogous to the considerations explained above in relation to the first time period.

The determining the probability of the subject having the severe acute respiratory syndrome may, for example, be partially based on a change in the minimum or average peak inhalation flow relative to a baseline peak inhalation flow.

For enhanced accuracy in determining the probability, the change in the minimum or average peak inhalation flow relative to the baseline may be, for instance, 10% or more, such as 50% or more or 90% or more. The baseline may, for example, be determined using daily minimum peak inhalation flows measured over a period in which no exacerbation has taken place and the subject is not suspected of having a severe acute respiratory syndrome, for example for 1 to 20 days. Alternatively or additionally, the minimum or average peak inhalation flow may be assessed relative to an absolute value.

The method 1020 may further comprise determining an inhalation volume, such as a minimum or average inhalation volume from inhalation volumes measured for inhalations performed during a third time period. The term “third” in relation to the third time period is to distinguish the period for sampling the inhalation volumes from the first time period during which the number of rescue inhalations are sampled, and the second time period during which the peak inhalation flow data are sampled. The third period may at least partially overlap with the first time period and/or the second time period, or the third time period may be concurrent with at least one of the first time period and the second time period.

The step 1026 of determining the probability may thus be partially based on the minimum or average inhalation volume. The third time period may be, for instance, 1 to 5 days, such as 1 day. The third time period may be selected according to the time required to gather minimum inhalation volume data of suitable indicative value, in a manner analogous to the considerations explained above in relation to the first time period.

The determining the probability may, for example, be partially based on a change in the minimum or average inhalation volume relative to a baseline inhalation volume.

For enhanced accuracy in determining the probability of the severe acute respiratory syndrome, the change in the minimum or average inhalation volume relative to the baseline may be, for instance, 10% or more, such as 50% or more or 90% or more. The baseline may, for example, be determined using daily minimum inhalation volumes measured over a period in which no exacerbation has taken place and the subject is not suspected of having a severe acute respiratory syndrome, for example for 1 to 10 days. Alternatively or additionally, the minimum or average inhalation volume may be assessed relative to an absolute value.

The method 1020 may further comprise determining an inhalation duration, such as a minimum or average inhalation duration from inhalation durations measured for inhalations over a fourth time period. The term “fourth” in relation to the fourth time period is to distinguish the period for sampling the minimum inhalation durations from the first time period during which the number of rescue inhalations are sampled, the second time period during which the peak inhalation flow data are sampled, and the third time period during which the inhalation volume data are sampled. The fourth time period may at least partially overlap with the first time period, the second time period and/or the third time period, or the fourth time period may be concurrent with at least one of the first time period, the second time period and the third time period.

The step 1026 of determining the probability may thus be partially based on the minimum or average inhalation duration. The fourth time period may be, for instance, 1 to 5 days, such as 1 day. The fourth time period may be selected according to the time required to gather minimum inhalation duration data of suitable indicative value, in a manner analogous to the considerations explained above in relation to the first time period.

The determining the probability may, for example, be partially based on a change in the minimum or average inhalation duration relative to a baseline inhalation duration.

For enhanced accuracy in determining the probability of the severe acute respiratory syndrome, the change in the minimum or average inhalation duration relative to the baseline may be, for instance, 10% or more, such as 50% or more or 90% or more. The baseline may, for example, be determined using daily minimum inhalation durations measured over a period in which no exacerbation has taken place and the subject is not suspected of having a severe acute respiratory syndrome, for example for 1 to 20 days. Alternatively or additionally, the minimum or average inhalation duration may be assessed relative to an absolute value.

FIGS. 8-12, described above, provide a non-limiting example of an inhaler which may be included in the system 1010. The terms processor 1014 and processing module 34 can be used interchangeably.

With regard to FIG. 11, in addition to the disclosure above, the slider 140 and switch 130 together with the electronics module 120 may correspond to a non-limiting example of the use determination system 1012B.

The sensor system 128 may be an example of the sensor system 1012A.

The no inhalation event may be associated with pressure measurement readings and/or airflow metrics below a particular threshold, such as an airflow rate less than 30 Lpm. The no inhalation event may occur when a subject does not inhale from the mouthpiece 106 after opening the mouthpiece cover 108 and during the measurement cycle. The no inhalation event may also occur when the subject's inspiratory effort is insufficient to ensure proper delivery of the medication via the flow pathway 119, such as when the inspiratory effort generates insufficient airflow to activate the deagglomerator 121 and, thus, aerosolize the medication in the dosing cup 116.

The low inhalation event may occur when the subject inhales from the mouthpiece 106 after opening the mouthpiece cover 108 and the subject's inspiratory effort causes at least a partial dose of the medication to be delivered via the flow pathway 119. That is, the inhalation may be sufficient to activate the deagglomerator 121 such that at least a portion of the medication is aerosolized from the dosing cup 116.

The good inhalation event may be associated with pressure measurement readings and/or airflow metrics above the low inhalation event, such as an airflow rate between 45 Lpm and 200 Lpm. The good inhalation event may occur when the subject inhales from the mouthpiece 106 after opening the mouthpiece cover 108 and the subject's inspiratory effort is sufficient to ensure proper delivery of the medication via the flow pathway 119, such as when the inspiratory effort generates sufficient airflow to activate the deagglomerator 121 and aerosolize a full dose of medication in the dosing cup 116.

Any suitable thresholds or ranges may be used to categorize a particular event. Some or all of the events may be used. For example, the no inhalation event may be associated with an airflow rate below 45 Lpm and the good inhalation event may be associated with an airflow rate between 45 Lpm and 200 Lpm. As such, the low inhalation event may not be used at all in some cases.

The personalized data collected from, or calculated based on, the usage of the inhaler 100 (e.g. pressure metrics, airflow metrics, lung function metrics, dose confirmation information, etc.) may be computed and/or assessed via external devices as well (e.g. partially or entirely). More specifically, the wireless communication circuit 129 in the electronics module 120 may include a transmitter and/or receiver (e.g. a transceiver), as well as additional circuitry. For example, the wireless communication circuit 129 may include a Bluetooth chip set (e.g. a Bluetooth Low Energy chip set), a ZigBee chipset, a Thread chipset, etc. As such, the electronics module 120 may wirelessly provide the personalized data, such as pressure measurements, airflow metrics, lung function metrics, dose confirmation information, and/or other conditions related to usage of the inhaler 100, to an external device, including a smart phone. The personalized data may be provided in real time to the external device to enable the above-described probability determination based on real-time data from the inhaler 100 that indicates time of use, how the inhaler 100 is being used, and personalized data about the user of the inhaler, such as real-time data related to the subject's lung function and/or medical treatment. The external device may include software for processing the received information and for providing compliance and adherence feedback to users of the inhaler 100 via a graphical user interface (GUI).

### Embodiments

The application also includes the following embodiments:
[1]. A system for determining a probability that a subject having a chronic respiratory disease also has a severe acute respiratory syndrome, the system comprising:
a first inhaler for delivering a rescue medicament, the first inhaler having a use determination system configured to determine a rescue inhalation performed by the subject using the first inhaler;
an optional second inhaler for delivering a maintenance medicament to the subject during a routine inhalation,
wherein the system comprises a sensor system configured to measure a parameter relating to airflow during said rescue inhalation and/or during said routine inhalation using the second inhaler when included in the system; and
a processor configured to:
determine a number of said rescue inhalations during a first time period;
receive said parameter measured for at least some of said rescue and/or routine inhalations; and
determine said probability of the subject having the severe acute respiratory syndrome based on said number of rescue inhalations and said parameters.
[2]. The system according to embodiment [1], wherein the chronic respiratory disease is asthma, and wherein the processor is configured to determine said probability using a weighted model which is weighted such that the number of rescue inhalations is more significant in the probability determination than said parameter.
[3]. The system according to embodiment [1], wherein the chronic respiratory disease is chronic obstructive pulmonary disease, and wherein the processor is configured to determine said probability using a weighted model which is weighted such that the parameter is more significant in the probability determination than said number of rescue inhalations.
[4]. The system according to any of embodiments [1] to [3], wherein the rescue medicament is albuterol.
[5]. The system according to any of embodiments [1] to [4], wherein the parameter is at least one of a peak inhalation flow, an inhalation volume and an inhalation duration.
[6]. The system according to embodiment [5], wherein the processor is further configured to determine a minimum peak inhalation flow from peak inhalation flows measured for rescue and/or routine inhalations performed during a second time period, and wherein the probability is partially based on said minimum peak inhalation flow; optionally wherein the second time period is 1 to 5 days.
[7]. The system according to embodiment [6], wherein the processor is configured to determine said probability based on a change in the minimum peak inhalation flow relative to a baseline peak inhalation flow.
[8]. The system according to any of embodiments [5] to [7], wherein the processor is further configured to determine a minimum inhalation volume from inhalation volumes measured for rescue and/or routine inhalations performed during a third time period, and wherein the probability is partially based on said minimum inhalation volume; optionally wherein the third time period is 1 to 5 days.
[9]. The system according to embodiment [8], wherein the processor is configured to determine said probability partially based on a change in the minimum inhalation volume relative to a baseline inhalation volume.
[10]. The system according to any of embodiments [5] to [9], wherein the processor is further configured to determine a minimum inhalation duration from inhalation durations measured for rescue and/or routine inhalations over a fourth time period, and wherein the probability is partially based on said minimum inhalation duration; optionally wherein the fourth time period is 1 to 5 days.
[11]. The system according to embodiment [10], wherein the processor is configured to determine said probability partially based on a change in the minimum inhalation duration relative to a baseline inhalation duration.
[12]. The system according to any of embodiments [1] to [11], wherein the sensor system comprises a pressure sensor; optionally wherein the use determination system comprises a further pressure sensor, the pressure sensor and the further pressure sensor being the same as or different from each other.
[13]. The system according to any of embodiments [1] to [12], wherein the first inhaler comprises:
a medicament reservoir; and
a dose metering assembly configured to meter a dose of said medicament from the reservoir, wherein the use determination system is configured to register the metering of said dose by the dose metering assembly, each metering being thereby indicative of said rescue inhalation performed by the subject using the first inhaler.
[14]. The system according to any of embodiments [1] to [13], further comprising a user interface for inputting an indication of a status of the chronic respiratory disease being experienced by the subject, wherein the processor is configured to determine, using said weighted model, said probability based on said number of rescue inhalations, said parameters, and said received indication.
[15]. A method for determining a probability that a subject having a chronic respiratory disease also has a severe acute respiratory syndrome, the method comprising:
receiving a number of rescue inhalations of a rescue medicament performed by the subject during a first time period;
receiving a parameter relating to airflow during at least some of the rescue inhalations and/or during routine inhalations performed by the subject of a maintenance medicament; and
determining said probability of the subject having the severe acute respiratory syndrome based on said number of rescue inhalations and said parameters.
[16]. The method according to embodiment [15], wherein the chronic respiratory disease is asthma, and wherein the processor is configured to determine said probability using a weighted model which is weighted such that the number of rescue inhalations is more significant in the probability determination than said parameter.
[17]. The method according to embodiment [15], wherein the chronic respiratory disease is chronic obstructive pulmonary disease, and wherein the processor is configured to determine said probability using a weighted model which is weighted such that the parameter is more significant in the probability determination than said number of rescue inhalations.
[18]. The method according to any of embodiments [15] to [17], wherein the method further comprises providing a first inhaler for delivering said rescue medicament to the subject, the first inhaler having a use determination system configured to determine said rescue inhalation performed by the subject using the first inhaler.
[19]. The method according to any of embodiments [15] to [18], wherein the method further comprises providing a sensor system configured to measure said parameter relating to airflow during said rescue inhalation and/or said routine inhalation.
[20]. A computer program comprising computer program code which is adapted, when said program is run on a computer, to implement the method of any of embodiments [15] to [17].
[21]. A method for treating a severe acute respiratory syndrome in a subject suffering from a chronic respiratory disease, the method comprising:
performing the method according to any of embodiments [15] to [19];
determining whether the probability reaches or exceeds a predetermined upper threshold; or
determining whether the probability reaches or is lower than a predetermined lower threshold; and
treating said severe acute respiratory syndrome based on said probability reaching or exceeding the predetermined upper threshold; or based on said probability reaching or being lower than said predetermined lower threshold.
[22]. The method according to embodiment [21], wherein the treating comprises switching the subject from a first treatment regimen to a second treatment regimen based on said probability reaching or exceeding the predetermined upper threshold, wherein the second treatment regimen is configured for higher risk of the severe acute respiratory syndrome than said first treatment regimen.
[23]. The method according to embodiment [22], wherein the second treatment regimen comprises admitting the subject into an intensive care unit and/or administering dexamethasone to the subject.
[24]. The method according to embodiment [21], wherein the treating comprises switching the subject from a first treatment regimen to a third treatment regimen based on said probability reaching or being lower than the predetermined lower threshold, wherein the third treatment regimen is configured for lower risk of the severe acute respiratory syndrome than said first treatment regimen; optionally wherein the first treatment regimen comprises treating the subject in an intensive care unit, and the third treatment regimen comprises treating the subject in a setting which is outside the intensive care unit.
[25]. A method for diagnosing a severe acute respiratory syndrome the method comprising:
performing the method according to any of embodiments [15] to [19];
determining whether the probability reaches or exceeds a predetermined upper threshold indicative of the severe acute respiratory syndrome; and
diagnosing said severe acute respiratory syndrome based on said probability reaching or exceeding the predetermined upper threshold.

What is claimed is:

1. A system for determining a level of acute risk to a subject suffering from a chronic respiratory disease, the system comprising:
an inhaler configured to deliver a medicament to the subject for treating the respiratory disease, the inhaler comprising a use determination system configured to determine at least one first value of a usage parameter relating to use of the inhaler; and
a processor configured to:
receive the at least one first value;
receive a second value indicative of whether the subject is also suffering from a viral respiratory infection; and
determine said level of acute risk based on the at least one first value and the second value.

2. The system according to claim 1, comprising a user interface, wherein the processor is configured to control the user interface to issue a notification based on said level of risk or wherein the user interface is configured to enable input of said second value.

3. The system according to claim 2, wherein the user interface is at least partly defined by a first user interface of a user device; and
wherein the user device is at least one selected from a personal computer, a tablet computer, and a smart phone.

4. The system according to claim 3, wherein the processor is at least partly included in a first processor included in the user device.

5. The system according to claim 1, wherein the second value is a value obtained from a virus detection method for detecting the virus of the respiratory viral infection in a sample from the subject; and
wherein the second value is a positive or a negative result from the virus detection method.

6. The system according to claim 5, wherein said virus detection method is at least one of an assay configured for selective binding of the virus, or a nucleic acid detection method for detecting at least part of the viral genome of the virus.

7. The system according to claim 1, wherein the usage parameter comprises a use of the inhaler; and
wherein the use determination system comprises a sensor for detecting inhalation of the respective medicament performed by the subject or a mechanical switch configured to be actuated prior to, during, or after use of the inhaler.

8. The system according to claim 1, wherein the usage parameter comprises a parameter relating to airflow during inhalation of the respective medicament performed by the subject.

9. The system according to claim 8, wherein the use determination system comprises a sensor for sensing the parameter; and
wherein the parameter is at least one of a peak inhalation flow, an inhalation volume, a time to peak inhalation flow, or an inhalation duration.

10. The system according to claim 9, wherein the sensor for sensing the parameter is the same as or different from the sensor for detecting inhalation of the respective medicament performed by the subject.

11. The system according to claim 1, wherein the medicament comprises albuterol, budesonide, beclomethasone, fluticasone, formoterol, salmeterol, indacaterol, vilanterol, tiotropium, aclidinium, umeclidinium, glycopyrronium, salmeterol combined with fluticasone, beclomethasone combined with albuterol, or budesonide combined with formoterol.

12. The system according to claim 1, wherein the viral respiratory infection is caused by a rhinovirus, an influenza virus, a respiratory syncytial virus, an adenovirus, a metapneumovirus, or a coronavirus.

13. A method for determining a level of acute risk to a subject suffering from a chronic respiratory disease, the method comprising:
receiving at least one first value of a usage parameter relating to use of an inhaler from a use determination system included in an inhaler configured to deliver a medicament to the subject for treating the respiratory disease;
receiving a second value indicative of whether the subject is also suffering from a viral respiratory infection; and
determining said level of acute risk based on the at least one first value and the second value.

14. The method according to claim 13, further comprising controlling a user interface to issue a notification based on said level of risk or wherein the receiving the second value comprises receiving the second value inputted via a user interface.

15. The method according to claim 13, wherein the second value is a value obtained from a virus detection method for detecting the virus of the respiratory viral infection in a sample from the subject; and
wherein the second value is a positive or a negative result from the virus detection method.

16. The method according to claim 15, wherein said virus detection method is at least one of an assay configured for selective binding of the virus, or a nucleic acid detection method for detecting at least part of the viral genome of the virus.

17. The method according to claim 13, wherein the usage parameter comprises a use of the inhaler or a parameter relating to airflow during an inhalation performed by the subject during the use of the inhaler.

18. The method according to claim 13, wherein the viral respiratory infection is caused by a rhinovirus, an influenza virus, a respiratory syncytial virus, an adenovirus, a metapneumovirus, or a coronavirus.

19. (canceled)

20. A computer-readable storage medium comprising computer-executable instructions that, when executed by a processor, cause the processor to:
receive at least one first value of a usage parameter relating to use of an inhaler from a use determination system included in an inhaler configured to deliver a medicament to the subject for treating the respiratory disease;
receive a second value indicative of whether the subject is also suffering from a viral respiratory infection; and
determine said level of acute risk based on the at least one first value and the second value.

21. The computer-readable storage medium according to claim 20, wherein, when executed by the processor, cause the processor to:
control a user interface to issue a notification based on said level of risk or wherein the receiving the second value comprises receiving the second value inputted via a user interface;
wherein the second value is a value obtained from a virus detection method for detecting the virus of the respiratory viral infection in a sample from the subject; and
wherein the second value is a positive or a negative result from the virus detection method.
